(19) United States
(12) Patent Application Publication
(10) Pub. No.: US 2025/0305060 A1
(43) Pub. Date: Oct. 2, 2025

(54) POLE VARIANT CLASSIFICATION STRATEGY IDENTIFIES PATIENTS WHO MAY HAVE A FAVORABLE PROGNOSIS AND BENEFIT FROM IMMUNOTHERAPY

(71) Applicant: Foundation Medicine, Inc., Boston, MA (US)

(72) Inventors: Rachel B. EVANS, Cambridge, MA (US); Brennan DECKER, Winchester, MA (US); Zoe R. FLEISCHMANN, Jamaica Plain, MA (US); Smruthy K. SIVAKUMAR, Everett, MA (US)

(73) Assignee: Foundation Medicine, Inc., Boston, MA (US)

(21) Appl. No.: 19/091,397

(22) Filed: Mar. 26, 2025

Related U.S. Application Data
(60) Provisional application No. 63/571,402, filed on Mar. 28, 2024.

(51) Int. Cl. C12Q 1/6886; C12Q 1/6855

(52) U.S. Cl. CPC C12Q 1/6886; C12Q 1/6855; C12Q 2600/106; C12Q 2600/156

(57) ABSTRACT

Methods are described for identifying and/or treating patients who may benefit from immunotherapy based on classification of POLE variants identified using genomic profiling data. In some instances, for example, the disclosed methods of treating a subject having a cancer comprise: acquiring a genomic profile based on a sample from the subject, wherein the genomic profile is indicative of: (i) the presence of a pathogenic POLE (pPOLE) variant, or (ii) the presence of a pathogenic POLE (pPOLE) variant and a microsatellite instability (MSI) status of MSI-high; and responsive to an indication that a pathogenic POLE (pPOLE) variant is present, with or without an indication of MSI-high status, administering an anti-cancer therapy to the subject; thereby treating the subject.

### CROSS-REFERENCE TO RELATED APPLICATIONS

This application claims the priority benefit of U.S. Provisional Patent Application Ser. No. 63/571,402, filed Mar. 28, 2024, the contents of which are incorporated herein by reference in their entirety.

### FIELD

The present disclosure relates generally to methods for analyzing genomic profiling data, and more specifically to methods for identifying and/or treating patients who may benefit from immunotherapy based on classification of POLE variants identified using genomic profiling data.

### BACKGROUND

DNA polymerase epsilon (POLE) is encoded by the POLE gene and synthesizes the leading DNA strand during genome replication and cell division. Initial replication is highly mismatch prone and genomic integrity is dependent upon the proofreading and correction functions of the POLE exonuclease domain (ExoD) and downstream error mitigation mechanisms, including post-replicative mismatch repair (MMR). A subset of mutations involving the POLE ExoD disrupt replication fidelity, leading to transmission of uncorrected errors to daughter cells. Indeed, selected somatic POLE ExoD alterations (pathogenic POLE [pPOLE]), cause de novo mutations to arise with each successive mitotic cycle, leading to accumulation of abundant mutations manifested as a characteristic ultramutated tumor phenotype. This phenotype is defined by markedly elevated tumor mutational burden (TMB≥100 mut/Mb) and characteristic COSMIC single base substitution (SBS) mutational signatures (e.g., SBS10, SBS14). Somatic ultramutation defines a biologically distinct subset of malignancies and has been observed as a driver of oncogenesis or tumor evolution in numerous cancer types, most frequently endometrial (EC) and colorectal (CRC) carcinomas. Ultramutated tumors are of increasing biological and clinical significance because the plethora of alterations in these tumors represent potential neoantigens that may elicit anti-tumor immune responses when treated with immune checkpoint inhibitor (ICI) therapies. Further, pPOLE variants portend favorable prognosis independent of ICI treatment. Assessment of POLE status is recommended in the National Comprehensive Cancer Network (NCCN) guidelines for several cancer types, reflecting the recognized importance of this pan-tumor biomarker.

The clinical implications of pPOLE variants make accurate classification consequential. Historical classification of pPOLE variants has been anecdotal and based on limited evidence (e.g., localization to the ExoD with or without the context of elevated TMB).

### BRIEF SUMMARY OF THE INVENTION

Disclosed herein are methods for classification of POLE variants comprising a functional readout-based framework leveraging multiple features of the POLE-associated ultramutated tumor phenotype to assign pathogenicity status for both recurrent and rare POLE variants. Confident pathogenicity assignment enables nuanced investigation of the consequences of POLE exonuclease deficiency, revealing unique aspects of this phenomenon across cancer types, between specific pathogenic alleles, and in diverse clinical settings. In particular, the interaction between pPOLE and MMR-associated mutagenesis was explored in tumors exhibiting deficiency of both mechanisms. These observations enable POLE-associated mutagenesis to be ruled out in some cancers, and have implications for improved diagnosis and clinical management of patients with tumors harboring pPOLE.

Disclosed herein are methods for diagnosing or confirming a diagnosis of disease in a subject, comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and diagnosing or confirming a diagnosis of the disease in the subject based on an indication that the pathogenic POLE (pPOLE) variant is present, with and without an indication of MSI-high status. In some embodiments, the method further comprises administering a treatment for the disease to the subject based on the diagnosis or confirmation of a diagnosis of the disease.

Disclosed herein are methods for identifying a subject for treatment of a disease, comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and identifying the subject for treatment of the disease based on an indication that the pathogenic POLE (pPOLE) variant is present, with and without an indication of MSI-high status.

Disclosed herein are methods for predicting a treatment outcome for a subject having a disease, comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and predicting a treatment outcome for the subject based on an indication that the pathogenic POLE (pPOLE) variant is present, with and without an indication of MSI-high status.

Disclosed herein are methods for selecting a treatment for a subject having a disease, the methods comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and selecting a treatment for the subject based on an indication that the pathogenic POLE (pPOLE) variant is present, with and without an indication of MSI-high status.

Disclosed herein are methods of treating a subject having a disease, the methods comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and responsive to an indication that a pathogenic POLE (pPOLE) variant is present, with and without an indication of MSI-high status, administering a treatment for the disease to the subject; thereby treating the subject.

Disclosed herein are methods for adjusting a treatment dose for a subject having a disease, comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and responsive to an indication that a pathogenic POLE (pPOLE) variant is present, with and without an indication of MSI-high status, adjusting the treatment dose for the subject.

Disclosed herein are methods for monitoring disease progression or recurrence in a subject comprising: a) determining a first disease status indicator for the subject based on a genomic profile for a first sample obtained from the subject at a first time point, wherein the first disease status indicator indicates: (i) a presence of a pathogenic POLE (pPOLE) variant, or (ii) a presence of a pathogenic POLE (pPOLE) variant and a microsatellite instability (MSI) status of MSI-high; b) determining a second disease status indicator for the subject based on a genomic profile for a second sample obtained from the subject at a second time point, optionally wherein the second time point is after the subject has been treated for a disease, and wherein the second disease status indicator indicates: (i) a presence of the pathogenic POLE (pPOLE) variant, or (ii) a presence of the pathogenic POLE (pPOLE) variant and a microsatellite instability (MSI) status of MSI-high; c) comparing the second disease status indicator to the first disease status indicator; and d) determining, based on a change in disease status indicator that indicates the presence of the pPOLE variant in the second sample, that the disease is progressing or reoccurring, thereby monitoring the disease progression or recurrence. In some embodiments, the method further comprises determining a variant allele frequence (VAF) associated with the pPOLE variant if the pPOLE variant is determined to be present. In some embodiments, the method further comprises selecting a treatment for the disease in response to disease progression or recurrence. In some embodiments, the method further comprises administering the treatment to the subject in response to disease progression or recurrence. In some embodiments, the method further comprises making a decision to adjust the treatment for the subject in response to disease progression or recurrence. In some embodiments, the decision is a decision to select a different treatment in response to disease progression or recurrence. In some embodiments, the decision is a decision to keep the same treatment in response to disease progression or recurrence. In some embodiments, the method further comprises adjusting a dosage of the treatment in response to the disease progression or recurrence. In some embodiments, the method further comprises administering the adjusted treatment to the subject. In some embodiments, the first time point is before the subject has been treated for a disease, and wherein the second time point is after the subject has been treated for the disease. In some embodiments, the subject has a cancer, is at risk of having a cancer, is being routinely tested for cancer, or is suspected of having a cancer. In some embodiments, the cancer is a solid tumor. In some embodiments, the cancer is a hematological cancer.

In some embodiments, the disease is cancer. In some embodiments, the treatment comprises an anti-cancer therapy. In some embodiments, the anti-cancer therapy comprises chemotherapy, radiation therapy, immunotherapy, a targeted therapy, or surgery.

In some embodiments, the treatment comprises an immune checkpoint inhibitor. In some embodiments, the immune checkpoint inhibitor comprises an anti-PD-1 or anti-PD-L1 antibody. In some embodiments, the immune checkpoint inhibitor comprises Nivolumab, Pembrolizumab, Atezolizumab, Cemiplimab, Avelumab, Durvalumab, or any combination thereof.

In some embodiments, the cancer comprises an endometrial cancer, a colorectal cancer, a non-small cell lung cancer (NSCLC), a squamous NSCLC, a non-squamous NSCLC, a metastatic cutaneous squamous cell carcinoma, a small intestine adenocarcinoma, a glioma, or a metastatic Merkel cell carcinoma.

Also disclosed herein are methods for identifying a subject for inclusion in a clinical trial, the methods comprising: acquiring a genomic profile based on a sample from the subject, wherein the genomic profile is indicative of: (i) a presence of a pathogenic POLE (pPOLE) variant, or (ii) a presence of a pathogenic POLE (pPOLE) variant and a microsatellite instability (MSI) status of MSI-high; and identifying the subject as a candidate for inclusion in the clinical trial based on an indication that the pathogenic POLE (pPOLE) variant is present, with and without an indication of MSI-high status.

In some embodiments, an indication of the presence of a pathogenic POLE (pPOLE) variant in the genomic profile is based on an analysis of sequence read data derived from the sample from the subject. In some embodiments, the presence of a pathogenic POLE (pPOLE) variant is detected in the sequence read data using one or more processors and a variant calling algorithm. In some embodiments, the pPOLE variant comprises a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.

In some embodiments, an indication of MSI-high status in the genomic profile is based on an analysis of sequence read data for a plurality of microsatellite loci in the sample. In some embodiments, an indication of MSI-high status is indicative of a deficient DNA mismatch repair mechanism in the sample.

In some embodiments, the method further comprises applying an indication that a pPOLE variant is present in the sample as a diagnostic value associated with the sample.

In some embodiments, the genomic profile comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

In some embodiments, the sample is a tissue sample derived from the subject. In some embodiments, the sample is a liquid biopsy or hematological biopsy sample derived from the subject. In some embodiments, the sample is a liquid biopsy sample comprising blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA). In some embodiments, all or a portion of the cell-free DNA (cfDNA) comprises circulating tumor DNA (ctDNA).

Disclosed herein are methods comprising: providing a plurality of nucleic acid molecules obtained from a sample from a subject suspected of having or determined to have cancer; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; analyzing the sequence read data, using the one or more processors, to determine: (i) a presence of a pathogenic POLE (pPOLE) variant, or (ii) a presence of a pathogenic POLE (pPOLE) variant and a microsatellite instability (MSI) status of MSI-high; and responsive to an indication that a pathogenic POLE (pPOLE) variant is present, with and without an indication of MSI-high status, administering an anti-cancer therapy to the subject.

In some embodiments, the subject has a cancer, is at risk of having a cancer, is being routinely tested for cancer, or is suspected of having a cancer.

In some embodiments, the pPOLE variant comprises a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.

In some embodiments, the anti-cancer therapy comprises an immune checkpoint inhibitor. In some embodiments, the immune checkpoint inhibitor comprises an anti-PD-1 or anti-PD-L1 antibody. In some embodiments, the immune checkpoint inhibitor comprises Nivolumab, Pembrolizumab, Atezolizumab, Cemiplimab, Avelumab, Durvalumab, or any combination thereof.

In some embodiments, the cancer comprises an endometrial cancer, a colorectal cancer, a non-small cell lung cancer (NSCLC), a squamous NSCLC, a non-squamous NSCLC, a metastatic cutaneous squamous cell carcinoma, or a metastatic Merkel cell carcinoma.

In some embodiments, the cancer comprises a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the method further comprises obtaining the sample from the subject. In some embodiments, the sample comprises a tissue biopsy sample, a liquid biopsy sample, or a normal control. In some embodiments, the sample is a liquid biopsy sample and comprises blood, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA). In some embodiments, the cell-free DNA (cfDNA) or a portion thereof comprises circulating tumor DNA (ctDNA).

In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample.

In some embodiments, the sample comprises a liquid biopsy sample, and the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

In some embodiments, the method further comprises generating, by the one or more processors, a report indicating the presence or absence of a pathogenic POLE (pPOLE) variant and/or a microsatellite instability (MSI) status for the sample. In some embodiments, the method further comprises transmitting the report to a healthcare provider. In some embodiments, the report is transmitted via a computer network or a peer-to-peer connection.

In any of the embodiments disclosed herein, determination of microsatellite instability (MSI) status can comprise: identifying, using one or more processors, a set of microsatellite loci from a plurality of microsatellite loci based on a coverage requirement; applying, by the one or more processors, a set of sequence-based exclusion criteria to the set of microsatellite loci to identify a subset of the set of microsatellite loci; determining, by the one or more processors, a microsatellite instability (MSI) score for the sample based on a number of microsatellite loci in the set and a number of microsatellite loci in the subset; comparing, by the one or more processors, the MSI score to a predetermined threshold; and determining an MSI status of high microsatellite instability (MSI-high) for the sample if the MSI score is greater than or equal to the threshold.

In some embodiments, an indication of MSI-high status is indicative of a deficient DNA mismatch repair mechanism in the sample.

In some embodiments, the sample is a liquid biopsy sample or a hematological sample, and the plurality of microsatellite loci comprises at least 1,000 loci. In some embodiments, the sample is a tissue sample, and the plurality of microsatellite loci comprises at least 2,000 loci.

In some embodiments, the microsatellite loci comprise alleles having mononucleotide, dinucleotide, or trinucleotide repeat sequences.

In some embodiments, the sample is a tissue sample, and each microsatellite locus in the plurality of microsatellite loci comprises an allele having an overall length of at least 6 base pairs and less than 30 base pairs.

In some embodiments, each microsatellite locus in the plurality of microsatellite loci comprises an allele having a mononucleotide, dinucleotide, or trinucleotide repeat sequence at a minimum of 5× repeats, and having a total length of less than 50 base pairs.

In some embodiments, the coverage requirement is at least 75×, 100×, 150×, 150×, 200×, or 250×. In some embodiments, the coverage requirement is locus-dependent.

In some embodiments, applying the set of sequence-based exclusion criteria comprises excluding, from the set of microsatellite loci, a microsatellite locus that comprises an allele having an allele frequency below an allele frequency requirement. In some embodiments, the allele frequency requirement is at least 1%, at least 2%, at least 3%, at least 4%, at least 5%, at least 6%, at least 7%, at least 8%, at least 9%, or at least 10%.

In some embodiments, applying the set of sequence-based exclusion criteria comprises excluding, from the set of microsatellite loci, a microsatellite locus that comprises an erroneous allele sequence according to a statistical model.

In some embodiments, applying the set of sequence-based exclusion criteria comprises: comparing a particular allele at a particular microsatellite locus from the set of microsatellite loci to a reference database of sequencing errors; and excluding the particular microsatellite locus from the set of microsatellite loci if the particular allele corresponds to a known sequencing error. In some embodiments, the particular microsatellite locus is excluded if the particular allele is an allele of less than 10 base pairs in length and the particular allele has an allele frequency less than or equal to a mean allele frequency plus two standard deviations for the particular allele in the reference database of sequencing errors. In some embodiments, the particular microsatellite locus is excluded if the particular allele is an allele of greater than or equal to 10 base pairs in length and the particular allele has an allele frequency less than or equal to a mean allele frequency plus three standard deviations for the particular allele in the reference database of sequencing errors.

In some embodiments, applying the set of sequence-based exclusion criteria comprises comparing a particular allele at a particular microsatellite locus to one or more databases; and excluding the particular of microsatellite locus if the particular allele corresponds to a known germline allele.

In some embodiments, applying the set of sequence-based exclusion criteria comprises comparing a particular allele at a particular microsatellite locus to one or more databases; and excluding the particular microsatellite locus if the particular allele is equal in repeat length to a repeat length for the particular allele in the one or more databases, equal in overall length to an overall length for the particular allele in a reference human genome database, or equal in number of repeats to a number of repeats for the particular allele in the one or more databases.

In some embodiments, the set of sequence-based exclusion criteria is locus-dependent.

In some embodiments, the MSI score is calculated by comparing the number of microsatellite loci in the subset to the number of microsatellite loci in the set.

Also disclosed herein are systems comprising: one or more processors; and a memory communicatively coupled to the one or more processors and configured to store instructions that, when executed by the one or more processors, cause the system to perform any of the methods described herein.

Also disclosed herein are non-transitory computer-readable storage media storing one or more programs, the one or more programs comprising instructions, which when executed by one or more processors of a system, cause the system to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIGS. 1A-1F provide non-limiting examples of data for pathogenic POLE (pPOLE) variant classification and the landscape of unclassified POLE variants. FIG. 1A: plot of POLE variant samples per codon as a function of amino acid position. FIG. 1B: plot of POLE variant alleles per codon plotted as a function of amino acid position. FIG. 1C: plot of median TMB distribution of samples with unselected POLE variants as a function of amino acid position, and associated predominant mutational signatures. FIG. 1D: POLE-specific variant classification scheme applied to 497,769 TBx/LBx samples. FIG. 1E: plot illustrating the localization of pPOLE variants to the ExoD domain. FIG. 1F: plot of data illustrating the pan-tumor prevalence of unselected POLE and pPOLE variants.

FIGS. 2A-2E provide non-limiting examples of data that illustrates the association between pPOLE and ultramutation (TMB≥100Mut/Mb). FIG. 2A: Plot of TBx samples harboring pPOLE variants as a function of TMB. FIG. 2B: Plot of the TMB distribution of tumors stratified by POLE status in different cancer types. FIG. 2C: Plot of the TMB distribution of tumors harboring pPOLE variants versus bPOLE bariants versus bPOLE variants in the context of a non-POLE mutational signature. FIG. 2D: Plot of median TMB as a function of AlphaMissense pathogenicity score. FIG. 2E: Plot of the TMB distribution of tumors with pPOLE stratified by pPOLE VAF

FIGS. 3A-3D provide non-limiting examples of data that illustrates that pPOLE variants and microsatellite instability (MSI) are synergistic. FIG. 3A: Plot of allele-specific TMB and MSI status for selected variants. FIG. 3B: Plot of the proportion of pPOLE cases with concurrent MSI across select cancers. FIG. 3C: Plot of the TMB distribution of pPOLE and bPOLE tumors with or without co-occurring MSI. FIG. 3D: Plot of the number of SNV and indel mutations among samples with pPOLE, stratified by MSI status.

FIGS. 4A-4B provide non-limiting examples of data that illustrates the pPOLE co-mutational landscape for endometrial cancer (FIG. 4A), colorectal cancer (FIG. 4B).

FIG. 4C provides non-limiting examples of data for HRDsig score (left) and HRDsig status (right) of samples with or without pPOLE and with or without BRCA mutations.

FIG. 5 provides a non-limiting example of a POLE pan-tumor study cohort diagram.

FIGS. 6A-6D provide non-limiting examples of data that illustrate pathogenic POLE (pPOLE) variant classification and landscape. FIG. 6A: Plot of POLE variant alleles by allele type. FIG. 6B: Plot of the percentage of POLE variant alleles versus the number of observations across a pan-tumor cohort. FIG. 6C: Plot of the TMB distribution of samples exhibiting dominant mutational signatures in the pan-tumor cohort. FIG. 6D: Plot of the age distribution of patients with tumors harboring pPOLE in select cancers.

FIG. 7 provides non-limiting examples of data for MMR IHC/MSI PCR concordance in pPOLE+MSI samples, MMR IHC, MMR mutation status, and additional MSI testing results for pPOLE+MSI samples with available MMR IHC and/or MSI PCR data.

FIGS. 8A-8B provide non-limiting examples of data for paired sample analysis of tumors with pPOLE. FIG. 8A: pPOLE status and TMB of paired samples. FIG. 8B: clonal fraction (VAF/TP) of non-POLE variants either shared between or unique to individual samples across sample pairs.

FIG. 9 depicts an exemplary computing device or system in accordance with one embodiment of the present disclosure.

FIG. 10 depicts an exemplary computer system or computer network, in accordance with some instances of the systems described herein.

### DETAILED DESCRIPTION

Methods for classification of POLE variants are described that comprise a functional readout-based framework leveraging multiple features of the POLE-associated ultramutated tumor phenotype to determine the pathogenicity status for both individually recurrent and rare POLE variants. Confident pathogenicity assignment enables nuanced investigation of the consequences of POLE exonuclease deficiency, revealing unique aspects of this phenomenon across cancer types, between specific pathogenic alleles, and in diverse clinical settings. In particular, the interaction between pPOLE and MMR-associated mutagenesis was explored in tumors exhibiting deficiency of both mechanisms. These observations enable POLE-associated mutagenesis to be ruled out in some cancers, and have implications for the diagnosis and clinical management of patients with tumors harboring pPOLE.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” is a computationally generated sequence generated by a sequencer to represent a sequence of bases of a single strand of a sequenced fragment. A sequence read can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at 5′ and/or 3′ end), or any combination thereof.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods of Treating a Cancer Patient Based on Classification of POLE Variants

Methods of treating a subject (e.g., a cancer patient) based on classification of POLE variants identified in genomic profiling data for samples (e.g., tumor tissue samples, tumor tissue biopsy samples, or liquid biopsy samples) collected from the subject are described. The disclosed methods allow for more accurate classification of POLE variants identified in genomic profiling data as being pathogenic (pPOLE variants), and also allow one to identify samples for which tumor mutational burden (TMB) may be underestimated, thereby providing for more accurate identification of those individuals that are likely to respond to anti-cancer treatments such as immunotherapy treatments.

A POLE-specific phenotypic classification model encompassing tumor mutational burden (TMB), mutational signatures, germline frequency, and consideration of co-mutation with other POLE mutations was developed to identify pathogenic POLE (pPOLE) variants, e.g., selected somatic POLE ExoD alterations, that cause de novo mutations to arise with each successive mitotic cycle, leading to accumulation of abundant mutations manifested as a characteristic ultramutated tumor phenotype. This phenotype is defined by markedly elevated tumor mutational burden (TMB≥100 mut/Mb) 3 and characteristic COSMIC single base substitution (SBS) mutational signatures (e.g., SBS10, SBS14).

The POLE-specific phenotypic classification model comprises a functional readout-based framework leveraging multiple features of the POLE-associated ultramutated tumor phenotype to assign pathogenicity status for both recurrent and rare POLE variants. Twenty nine distinct pathogenic POLE variants (16 of which are novel pPOLE variants, see Table 2) were identified by applying this framework to a set of 7,404 distinct variant alleles identified in a; pan-cancer patient cohort. Pathogenic POLE (pPOLE) variants were identified by excluding: (i) germline variants in the gnomAD v2.1 exome database; (ii) variants with median TMB<20 mut/Mb across tumors; (iii) mutations associated with a POLE-associated SBS signature in less than half of samples in which they were observed; and (iv) variants that were only observed to co-occur with another pPOLE variant.

pPOLE variants were recurrent in cancers that have been previously associated with POLE-mediated ultramutation, such as those of the endometrium, colon and rectum, and nervous system. pPOLE variants were observed in both tissue biopsy (TBx) and liquid biopsy (LBx) samples, although the overall frequency was significantly higher in TBx which was likely due to low circulating tumor DNA shed and/or different clinical populations and ordering patterns for TBx versus LBx (including a decreased likelihood of collecting LBx samples in pPOLE patients due to favorable prognostic profiles).

In tissue samples with pPOLE variants present, the median TMB was 186.3 mut/Mb, which was significantly higher than the median TMB for all samples, as well as samples with any POLE variant present (3.5 and 10.0 mut/Mb, respectively). Among POLE variants within the ExoD, pPOLE variants were associated with a significantly higher TMB than bPOLE variants, including bPOLE variants observed in the context of a non-POLE mutational process (median TMB of 185.7 vs 4.4 and 27.8 mut/Mb, respectively). Compared to POLE wild-type samples, the presence of benign POLE (bPOLE) variants was associated with biological scenarios comprising numerous stochastic passenger mutations, including TMB-H, microsatellite instability-high (MSI-H), and mismatch repair-single base substitutions (MMR SBS).

Observations that pPOLE penetrance differed by variant allele and that it may depend on co-occurring mismatch repair deficiency led us to examine the interdependence of the pPOLE phenotype and MSI status. Co-occurrence of pPOLE variants and MSI produced a synergistic increase in TMB. TMB in MSI-H samples was approximately 2-fold higher than would result from an additive effect of pPOLE and MSI. Elevated TMB in tumors with a combined presence of pPOLE variants and MSI-H status was attributable to significant increases in both single base substitutions (SBS) and indels.

Though nearly all tumors with pPOLE variants present had high TMB, a small percentage had low TMB or no TMB estimate available. The latter included samples having significantly lower pPOLE variant allele fraction (VAF) compared to pPOLE samples with elevated TMB. Many of these samples also exhibited low tumor purity of <20%, and hence TMB was not clinically reported. These observations suggest that low tumor purity (i.e., near the limit of detection for TMB) can mask patients who may, in fact, have a high neoantigen load and would be expected to respond to ICI therapies, and also highlights the potential utility of detection of high specificity pPOLE variants as a standalone indicator of high TMB and/or patient response to ICI therapies.

Thus, the reliable classification of POLE variants as pathogenic (pPOLE) or benign (bPOLE) using the functional readout model described herein supports a change in clinical practice, where detection of a pPOLE variant can be interpreted as predicting ultramutation, favorable prognosis, and response to ICI-even when TMB status is not available as an orthogonal datapoint.

In some instances, the detection of one of the pPOLE variants disclosed herein (with or without a determination of TMB) may thus be used as genomic profiling-based biomarkers for: (i) diagnosing or confirming a diagnosis of disease (e.g., a cancer), (ii) identifying a subject (e.g., a patient) for treatment of a disease (e.g., a cancer), (iii) predicting a treatment outcome for a subject (e.g., a patient) having a disease (e.g., a cancer), (iv) selecting a treatment for a subject (e.g., a patient) having a disease (e.g., a cancer), (v) treating a subject (e.g., a patient) having a disease (e.g., a cancer), (vi) adjusting a treatment dose for a subject (e.g., a patient) having a disease (e.g., a cancer), (vii) monitoring disease (e.g., cancer) progression or recurrence in a subject (e.g., a patient), and/or identifying a subject (e.g., a patient) for inclusion in a clinical trial of a therapy (e.g., an anti-cancer therapy).

In some instances, for example, the disclosed methods of treating a subject having a disease (e.g., a cancer) may comprise: acquiring a genomic profile based on a sample from the subject, wherein the genomic profile is indicative of: (i) the presence of a pathogenic POLE (pPOLE) variant, or (ii) the presence of a pathogenic POLE (pPOLE) variant and a microsatellite instability (MSI) status of MSI-high (or mismatch repair deficient (MMRd)); and responsive to an indication that a pathogenic POLE (pPOLE) variant is present, with or without an indication of MSI-high status (or MMRd status), administering a therapy (e.g., an anti-cancer therapy) to the subject; thereby treating the subject.

In some instances, genomic profiling based on sequencing of nucleic acid molecules extracted from a sample from the subject may be performed as described elsewhere herein.

In some instances, determination of microsatellite instability (MSI) status (e.g., MSI-High, MSI-Low, and/or MSI-indeterminate) may be performed as described in PCT International Patent Application Publication No. WO 2023/287410, the contents of which are incorporated herein by reference in their entirety.

In some instances, a therapy may be an anti-cancer therapy. In some instances, the anti-cancer therapy may comprise an immune checkpoint inhibitor. In some instances, the immune checkpoint inhibitor may comprise, e.g., an anti-PD-1 or anti-PD-L1 antibody.

In some instances, the disease may be cancer. In some instances, the cancer may comprise, e.g., an endometrial cancer or a colorectal cancer.

In some instances, the pPOLE variant may be a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.

In some instances, the method may further comprise treating the cancer patient with an immune checkpoint inhibitor based on: (i) a determination that a pathogenic POLE (pPOLE) variant is present, or (ii) a determination that a pathogenic POLE (pPOLE) variant is present and the genomic data indicates a microsatellite instability (MSI) status for the sample of MSI-high.

In some instances, the immune checkpoint inhibitor comprises an anti-PD-1 or anti-PD-L1 antibody.

In some instances, the cancer may comprise an endometrial cancer or a colorectal cancer.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods may be used to select a subject (e.g., a patient) for a clinical trial based on the determination that a pathogenic POLE (pPOLE) variant is present in genomic data derived from a sample from the subject. In some instances, patient selection for clinical trials based on the methods described herein may accelerate the development of targeted therapies and improve the healthcare outcomes for treatment decisions.

In some instances, the disclosed methods for may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods may be used in treating a disease (e.g., a cancer) in a subject. For example, in response to determining a pPOLE or pPOLE plus MSI-High status for the subject using any of the methods disclosed herein, an effective amount of an anti-cancer therapy or anti-cancer treatment may be administered to the subject.

In some instances, the disclosed methods may be used for monitoring disease progression or recurrence (e.g., cancer or tumor progression or recurrence) in a subject. For example, in some instances, the methods may be used to determine pPOLE or pPOLE plus MSI-High status in a first sample obtained from the subject at a first time point, and used to determine pPOLE or pPOLE plus MSI-High status in a second sample obtained from the subject at a second time point, where comparison of the first determination and the second determination allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of pPOLE or pPOLE plus MSI-High status.

In some instances, the pPOLE or pPOLE plus MSI-High status determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining pPOLE or pPOLE plus MSI-High status as part of a genomic profiling process (or inclusion of the output from the disclosed methods for determining pPOLE or pPOLE plus MSI-High status as part of the genomic profile of the subject) can improve the validity of, e.g., disease detection calls and treatment decisions, made on the basis of the genomic profile by, for example, independently confirming the presence of a pPOLE variant or pPOLE plus MSI-High status in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly (A) tail of mature mRNAs by priming with oligo (dT)-containing oligonucleotides. Methods for depletion, poly (A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform “, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5):589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B.L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C.A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6):730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the disclosed methods. The systems may comprise, e.g., one or more processors, and a memory unit communicatively coupled to the one or more processors and configured to store instructions that, when executed by the one or more processors, cause the system to: receive sequence read data for a plurality of sequence reads derived from a sample from the subject, analyze the sequence read data to determine: (i) a presence of a pathogenic POLE (pPOLE) variant, or (ii) a presence of a pathogenic POLE (pPOLE) variant and a microsatellite instability (MSI) status of MSI-high; and responsive to an indication that a pathogenic POLE (pPOLE) variant is present, with and without an indication of MSI-high status, identify the subject as a candidate for anti-cancer therapy.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed methods and systems may be used for analysis of any of a variety of samples as described herein (e.g., a tissue sample, biopsy sample, hematological sample, or liquid biopsy sample derived from the subject).

In some instances, the plurality of gene loci for which sequencing data is processed to determine a presence of a plurality of somatic single base substitution variants may comprise at least 1, 2, 3, 4, 5, 6, 7, 8, 9, 10, 20, 30, 40, 50, 60, 70, 80, 0, 100, 200, 300, 400, 500, 600, 700, 800, 900, 1000, or more than 1000 gene loci (or any number of gene loci within the range of 1 to more than 1000 gene loci).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of pPOLE positive or pPOLE+MSI-High status is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

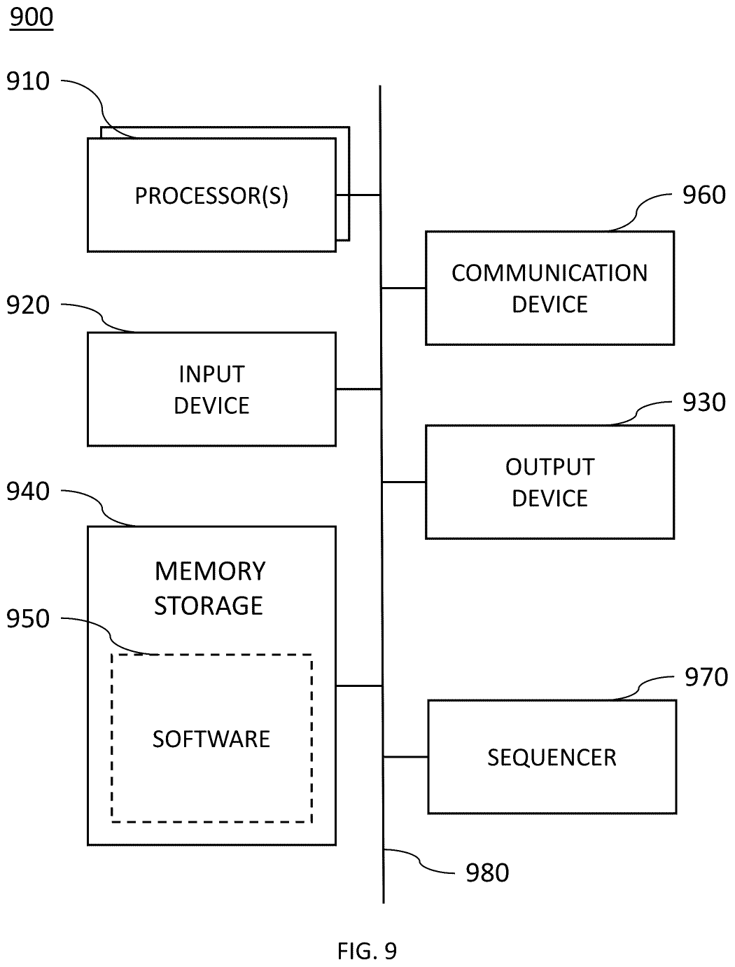

FIG. 9 illustrates an example of a computing device or system in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

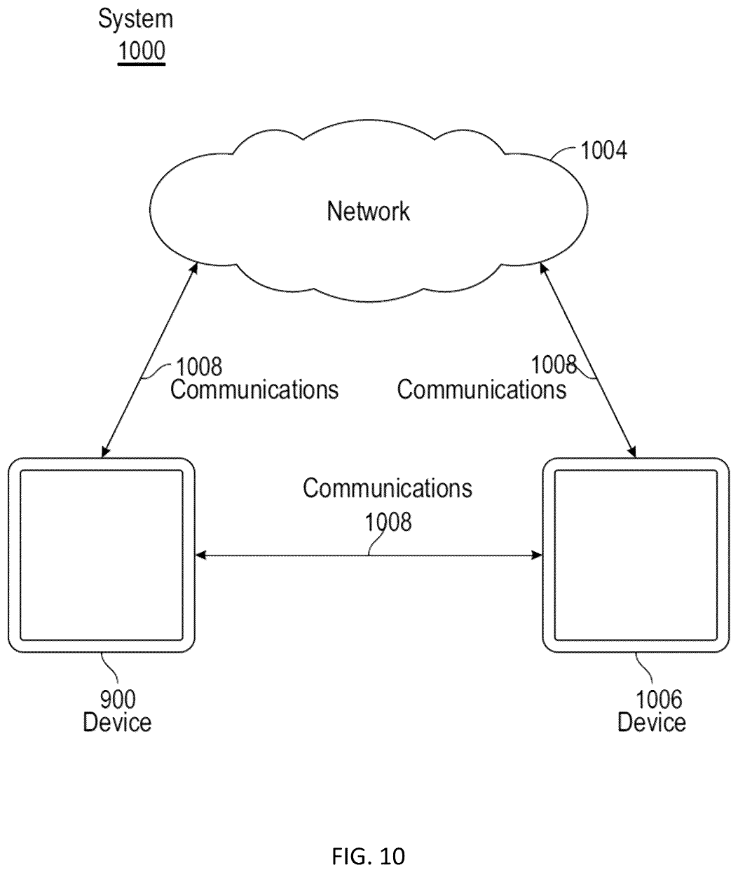

Device 900 may be connected to a network (e.g., network 1004, as shown in FIG. 10 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument.

FIG. 10 illustrates an example of a computing system in accordance with one embodiment. In system 1000, device 900 (e.g., as described above and illustrated in FIG. 9) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, Devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

### EXAMPLES

The following examples are included for illustrative purposes only and are not intended to limit the scope of the present disclosure.

### Example 1—Classification of POLE Variants

### I. Abstract

Pathogenic POLE (pPOLE) mutations undermine mismatch error correction by POLE during DNA replication and the resulting somatic ultramutation is predictive of response to immunotherapy. Historical classification of pPOLE is largely anecdotal and based on localization to the exonuclease domain (ExoD). A POLE-specific phenotypic classification model was developed that encompasses tumor mutational burden (TMB), mutational signatures, germline frequency, and consideration of co-mutation with other POLE mutations to identify pPOLE. The model was applied to >490,000 comprehensive genomic profiling (CGP) samples and identified 29 predicted pPOLE (including 29 novel pPOLE) out of 7,404 distinct POLE alleles (including 649 in the ExoD). pPOLE mutations were associated with ultramutation (median TMB≥100 mut/Mb) across tumor types. The synergistic association of pPOLE with microsatellite instability and clinical significance of pPOLE co-mutation landscapes was explored in endometrial and colorectal cancers. This study provides biological insight to guide classification and clinical management of patients with tumors harboring pPOLE.

### II. Methods

### Comprehensive Genomic Profiling

A US-based cohort of 497,769 patients who underwent CGP as part of routine clinical care was analyzed. In 455,965 TBx samples, CGP was performed on hybrid-captured, adaptor ligation-based libraries using DNA extracted from formalin-fixed paraffin-embedded (FFPE) tissue biopsies to interrogate up to 395 genes known to be associated with cancer, as previously described. In 41,804 LBx samples, CGP was performed using DNA extracted from whole blood samples to interrogate 324 cancer-associated genes, as previously described. CGP results were analyzed for mutations (base substitutions and short insertions/deletions), copy number changes (amplifications and homozygous deletions), and large genomic rearrangements, as well as complex biomarkers (see detailed methods below). Common germline polymorphisms and recurrent artifacts were removed, as previously described. Activating/likely activating alterations were called using a multi-step method leveraging annotations which include reporting in the Catalogue Of Somatic Mutations In Cancer (COSMIC), functional knowledge of the gene affected, internal insights, and clinical/functional characterization in the literature. Clonality of variants in TBx was determined using the ratio of the variant and sample estimated tumor fractions, with ≥50% considered clonal, based on all identified somatic mutations in a sample, as described previously. All CGP testing was performed in a Clinical Laboratory Improvement Amendments (CLIA)-certified, College of American Pathologists (CAP)-accredited, New York State-approved laboratory. Approval for this study, including a waiver of informed consent and a HIPAA waiver of authorization, was obtained from the Western Institutional Review Board (Protocol No. 20152817).

### Complex Biomarker Determination

Microsatellite instability (MSI) was determined from >2,000 repetitive loci. This method was previously validated on a cohort with matched mismatch repair (MMR) IHC and shown to be concordant in 273/279 (97.8%) samples. Tumor mutational burden (TMB) was determined, as previously described. Briefly, TMB was calculated using predicted somatic short variants across 0.8-1.1 Mb of sequenced DNA using a method that has been shown to be reflective of TMB in 29 matched normal samples sequenced by whole exome sequencing (R=0.74) and which demonstrated reproducibility in 60 replicate pairs (R=0.98). Homologous recombination deficiency signature (HRDsig) was determined using a copy number scar-based machine learning classifier.

### PD-L1 Immunohistochemistry

PD-L1 IHC was performed in a Clinical Laboratory Improvement Amendments (CLIA)-certified, College of American Pathologists (CAP)-accredited laboratory (Foundation Medicine, Inc., Morrisville, NC, USA). PD-L1 IHC was tested using the DAKO PD-L1 IHC 22C3 pharmDx assay which uses the mouse monoclonal 22C3 anti-PD-L1 clone. The assay was performed according to the package insert with appropriate controls. Scoring was performed by board-certified pathologists specifically trained in PD-L1 22C3 scoring.

### Statistical Analysis

For patient demographics, enrichment analysis compared pPOLE samples with POLE WT samples in all cases. The enrichment of TMB-H and differences in patient sex were evaluated with Fisher's Exact Test. Proportions of MSI status, genomic ancestry, and mutational signature categories were evaluated with Chi-Square. The difference in patient age distribution was evaluated with the Mann-Whitney U Test. Computation and plotting was carried out in using Python 3 (Python Software Foundation) and R 3.6.1 (R Foundation for Statistical Computing).

### III. Results

### Figure Legends and Tables

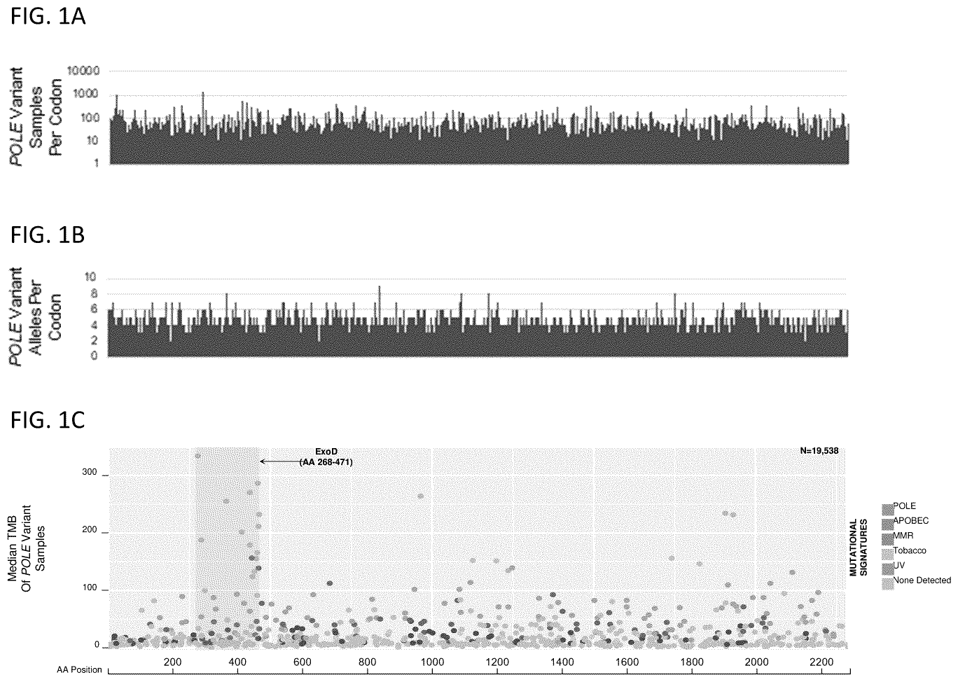

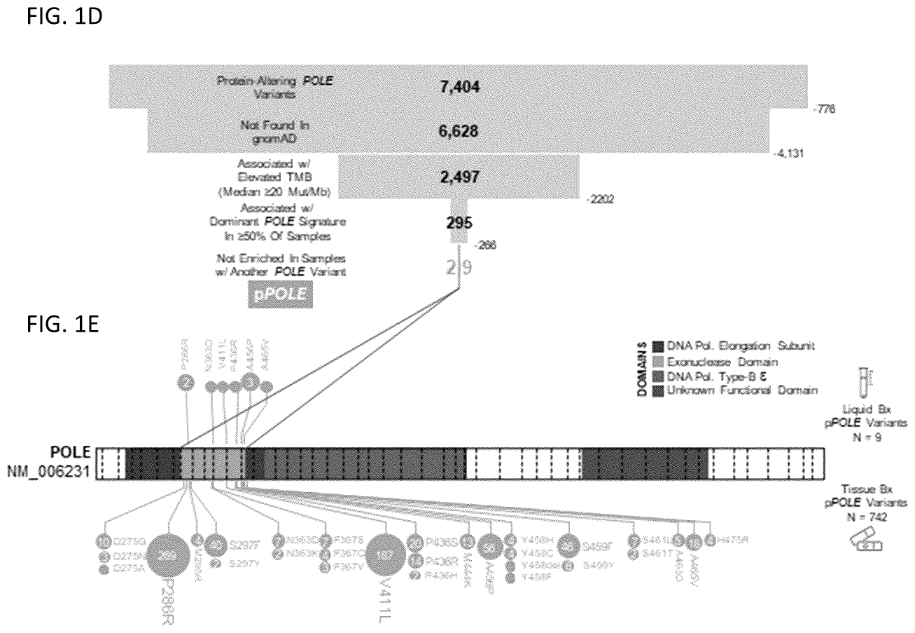

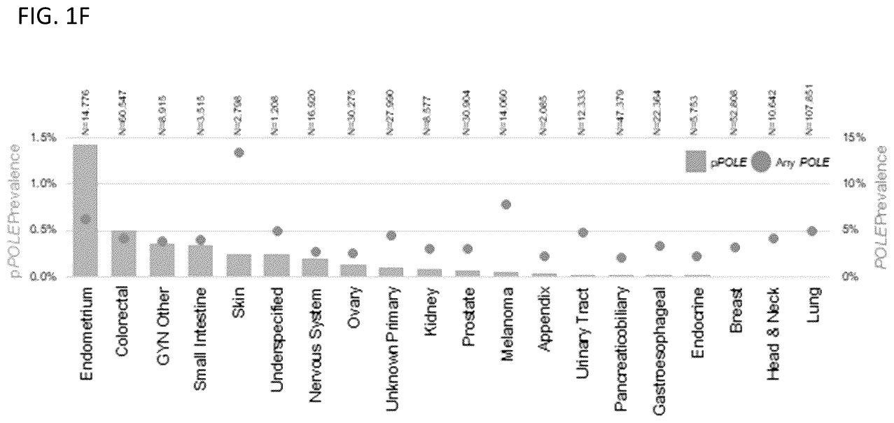

FIGS. 1A-1F. Pathogenic POLE (pPOLE) Variant Classification & Landscape Unclassified POLE. FIG. 1A: POLE variant samples per codon plotted as a function of amino acid position. FIG. 1B: POLE variant alleles per codon observed across 497,769 TBx/LBx samples plotted as a function of amino acid position. FIG. 1C: Median TMB distribution of samples with unselected POLE variants across the length of the POLE gene and associated predominant mutational signatures. FIG. 1D: POLE-specific variant classification scheme applied to 497,769 TBx/LBx samples. FIG. 1E: pPOLE variants (n=29) localize almost exclusively to the ExoD. The number of distinct pPOLE variants observed across TBx (n=742) and LBx (n=9) samples is indicated. FIG. 1F: Pan-tumor prevalence of unselected POLE (Gray Circles, Right Axis) and pPOLE (Teal Bars, Left Axis) variants. ExoD, Exonuclease Domain; LBx, Liquid Biopsy; TBx, Tissue Biopsy; TMB, Tumor Mutational Burden.

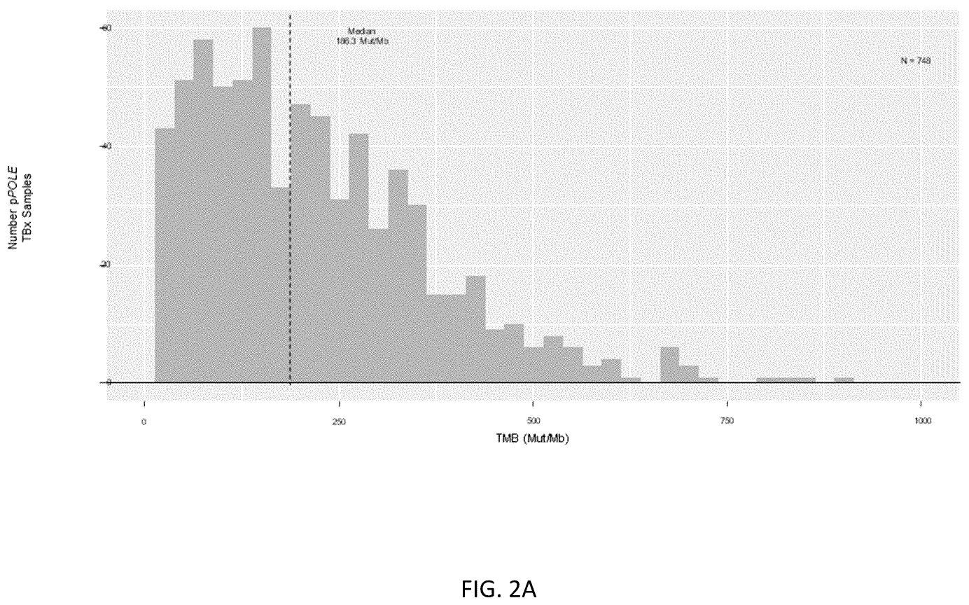

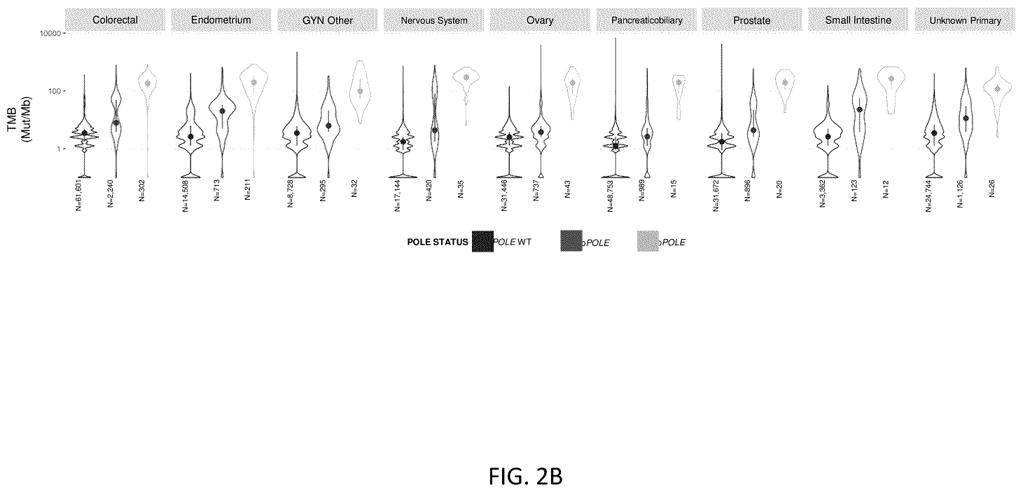

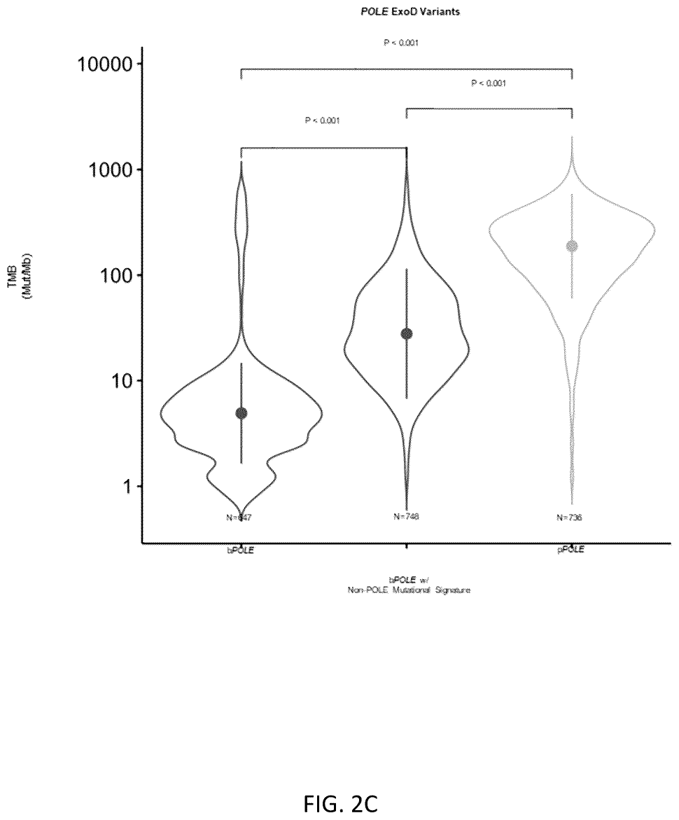

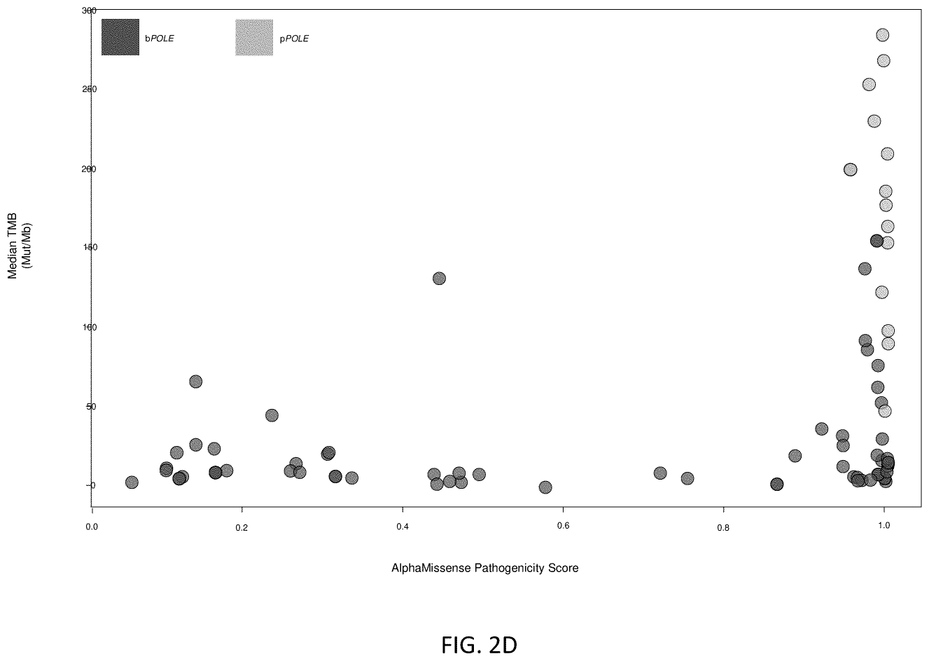

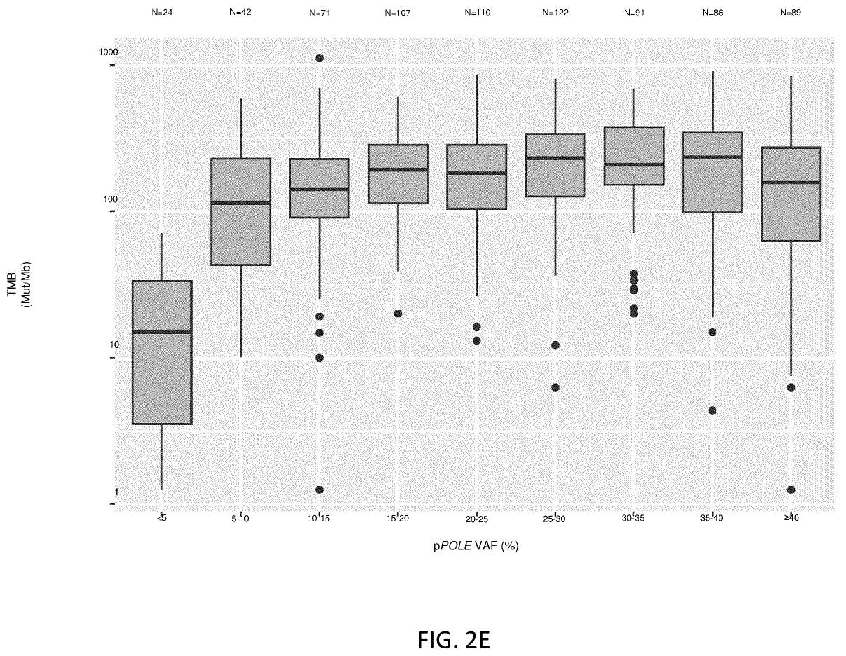

FIGS. 2A-2E. pPOLE are Associated with Ultramutation (TMB≥100Mut/Mb). FIG. 2A: TMB distribution of TBx samples harboring pPOLE variants (n=748). FIG. 2B: TMB distribution of tumors stratified by POLE status in cancer types with recurrent (>10) tumors with pPOLE. FIG. 2C: TMB distribution of tumors harboring pPOLE versus bPOLE versus bPOLE in the context of a non-POLE mutational signature. FIG. 2D: AlphaMissense pathogenicity scores and median TMB of variants classified as bPOLE or pPOLE. FIG. 2E: TMB distribution of tumors with pPOLE stratified by pPOLE VAF. bPOLE, Benign POLE; pPOLE, Pathogenic POLE; TMB, Tumor Mutational Burden; TBx, Tissue Biopsy; VAF, Variant Allele Fraction.

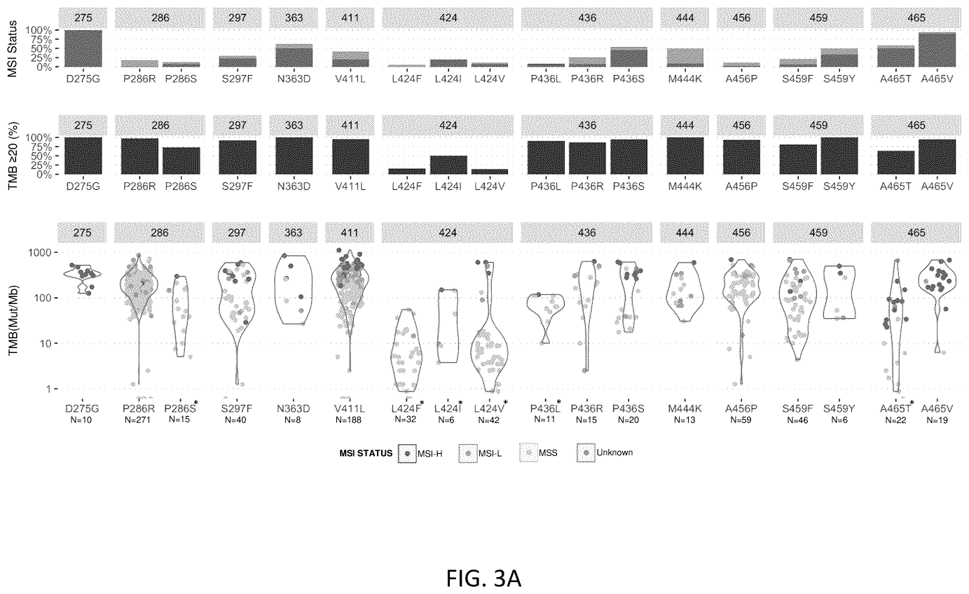

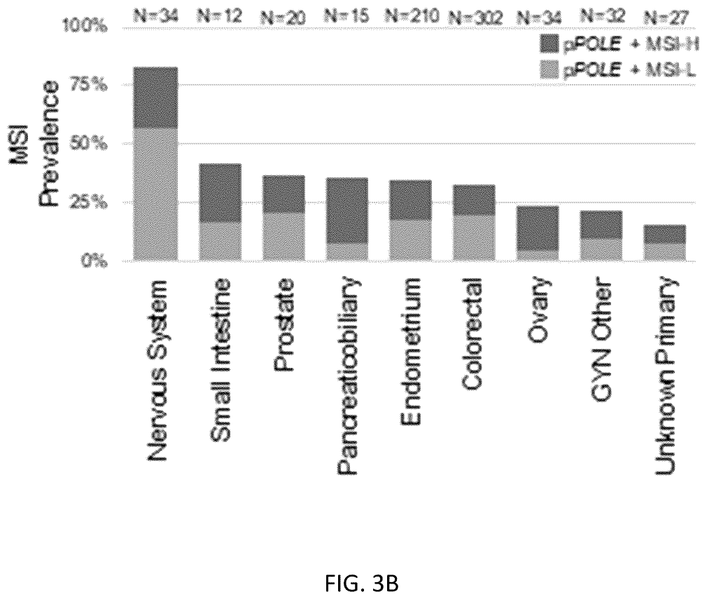

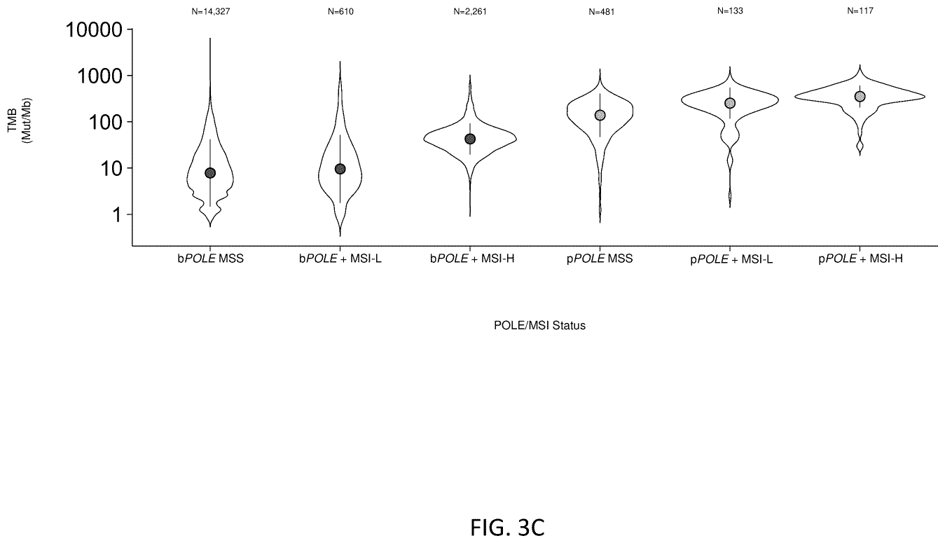

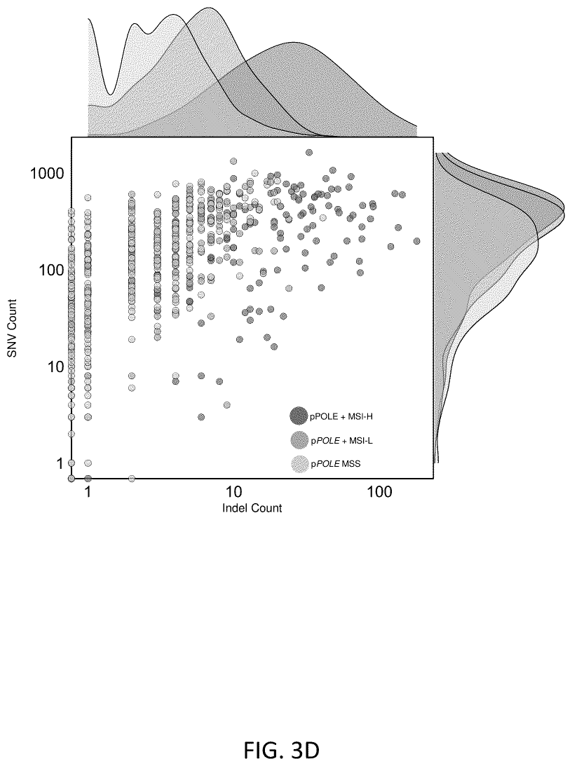

FIGS. 3A-3D. pPOLE+MSI are Synergistic. FIG. 3A: Allele-specific TMB and MSI status for selected variants, including pPOLE, bPOLE occurring at the same codon as a pPOLE, and variants at codon 424, which harbors p.L424V, a germline risk allele for polyposis and colorectal cancer. Only variants with ≥5 observations are shown. Asterisks (*) indicate bPOLE mutations. FIG. 3B: Proportion of pPOLE cases with concurrent MSI across select cancers. FIG. 3C: TMB distribution of pPOLE and bPOLE tumors with or without co-occurring MSI. FIG. 3D: Number of SNV and indel mutations among samples with pPOLE, stratified by MSI status. bPOLE, Benign POLE; Indel, Short Insertions/Deletions; MSI, Microsatellite Instability (Eq-Equivocal, H-High); MSS, Microsatellite Stable; pPOLE, Pathogenic POLE; SNV, Single Nucleotide Variants; TMB, Tumor Mutational Burden.

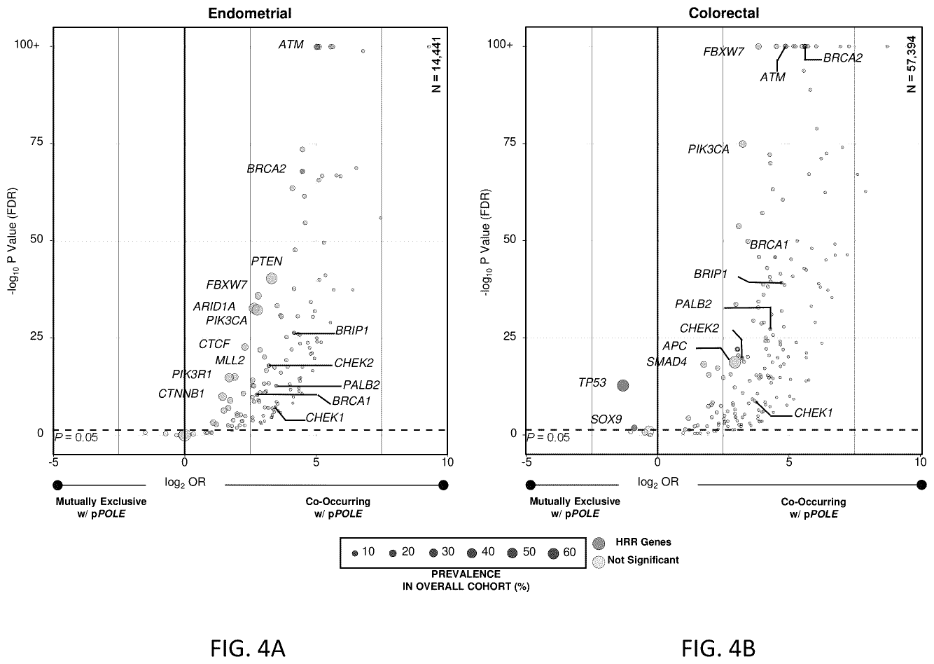

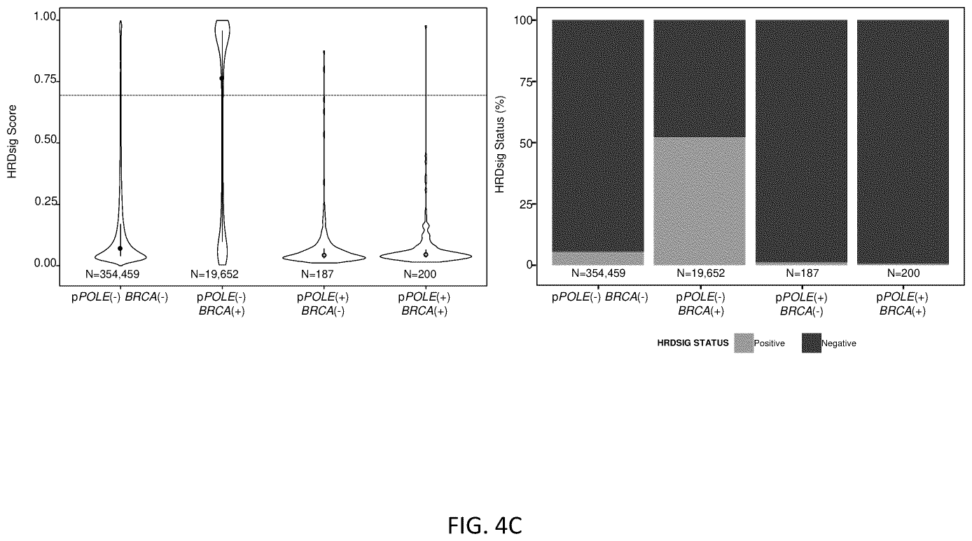

FIGS. 4A-4C. pPOLE Co-Mutation Landscape Volcano plots demonstrating gene-specific enrichment of mutations in pPOLE versus non-pPOLE endometrial (FIG. 4A) and colorectal (FIG. 4B) cancers. Only genes altered in at least 50 tumors and targeted across all tissue CGP assay versions were included. For each gene, only mutations (SNVs and indels) of known or likely functional significance were included. Genes altered at a high prevalence (≥10%) in either cohort are labeled for each volcano plot if a statistically significant difference was observed (p<0.05, threshold indicated by dashed line). HRR genes are highlighted in pink as an example of alterations that may have therapeutic associations. Fisher's Exact Test was performed to assess patterns of co-occurrence/mutual exclusivity between pPOLE and other genes. P values were corrected using the Benjamini-Hochberg FDR method. FIG. 4C: HRDsig score (Left) and HRDsig status (Right) of samples with or without pPOLE and with or without BRCA mutations. The HRDsig+/−threshold of 0.7 is indicated with a dashed line. HRDsig, Homologous Recombination Deficiency Signature; HRR, Homologous Recombination Repair; pPOLE, Pathogenic POLE; SNV, Single Nucleotide Variant.

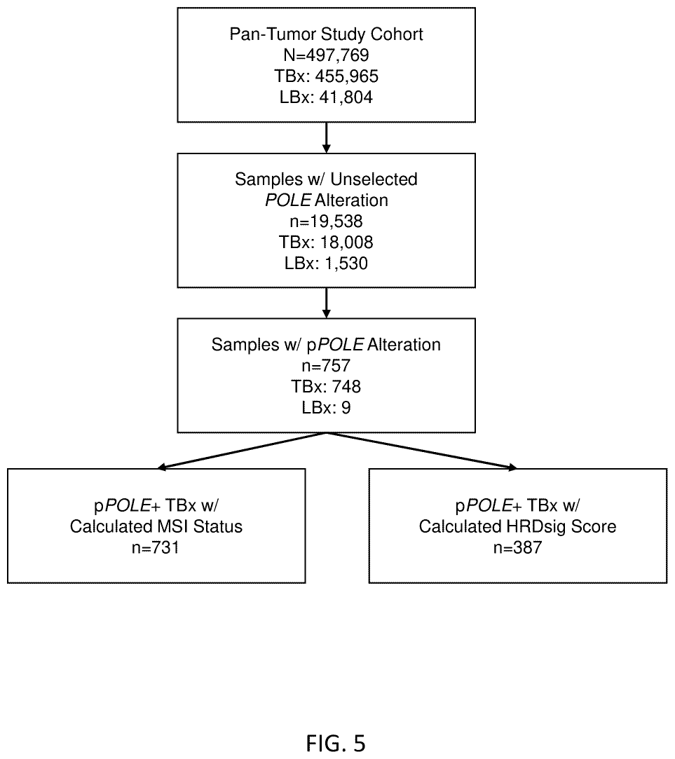

FIG. 5. POLE Pan-Tumor Study Cohort. Consort diagram showing the number of total samples examined in this study and the number available for specific biomarker analyses. HRDsig, Homologous Recombination Deficiency Signature; LBx, Liquid Biopsy Comprehensive Genomic Profiling; MSI, Microsatellite Instability; pPOLE, Pathogenic POLE; TBx, Tissue Biopsy Comprehensive Genomic Profiling.

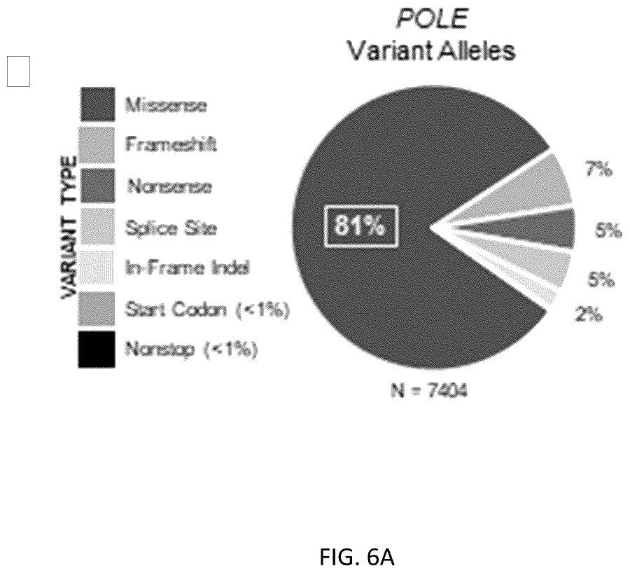

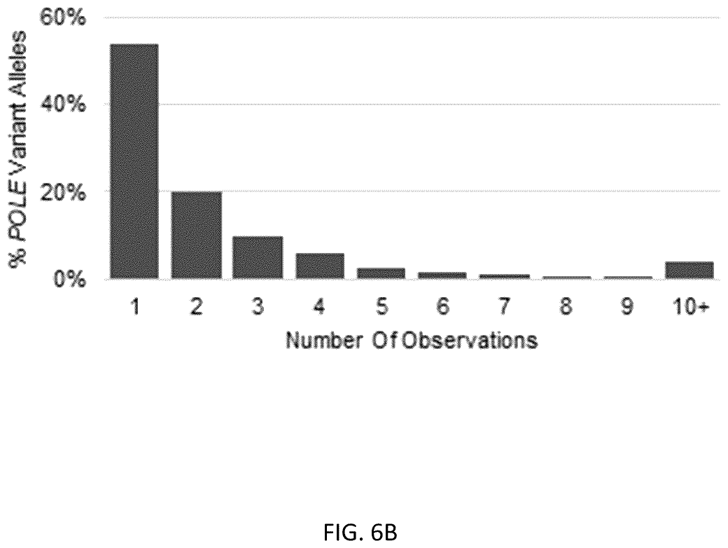

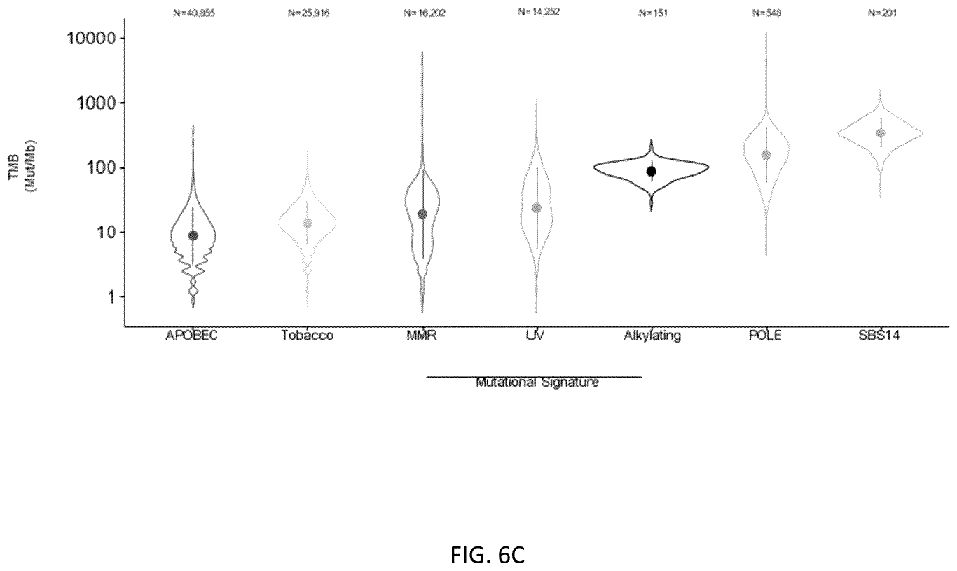

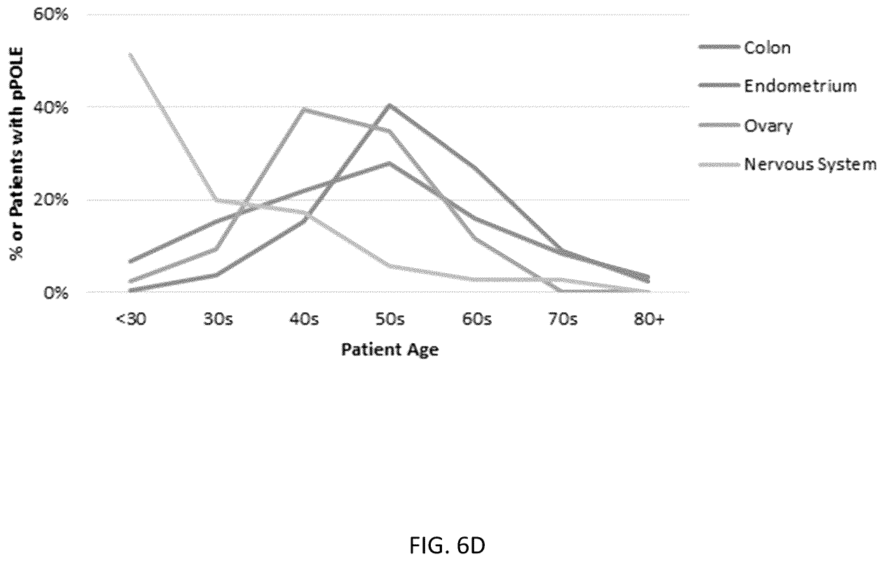

FIGS. 6A-6D. Pathogenic POLE (pPOLE) Variant Classification & Landscape—Additional Data. FIG. 6A: Unselected POLE variants represent 7,404 distinct alleles. FIG. 6B: Number of observations of unselected POLE alleles across the pan-tumor cohort. FIG. 6C: TMB distribution of samples exhibiting dominant mutational signatures in the pan-tumor cohort. FIG. 6D: Age distribution of patients with tumors harboring pPOLE in select cancers. APOBEC, Apolipoprotein B mRNA-Editing Enzyme, Catalytic Polypeptide; bPOLE, Benign POLE; ExoD, Exonuclease Domain; MMR, Mismatch Repair; pPOLE, Pathogenic POLE; SBS, Single Base Substitution Signature.

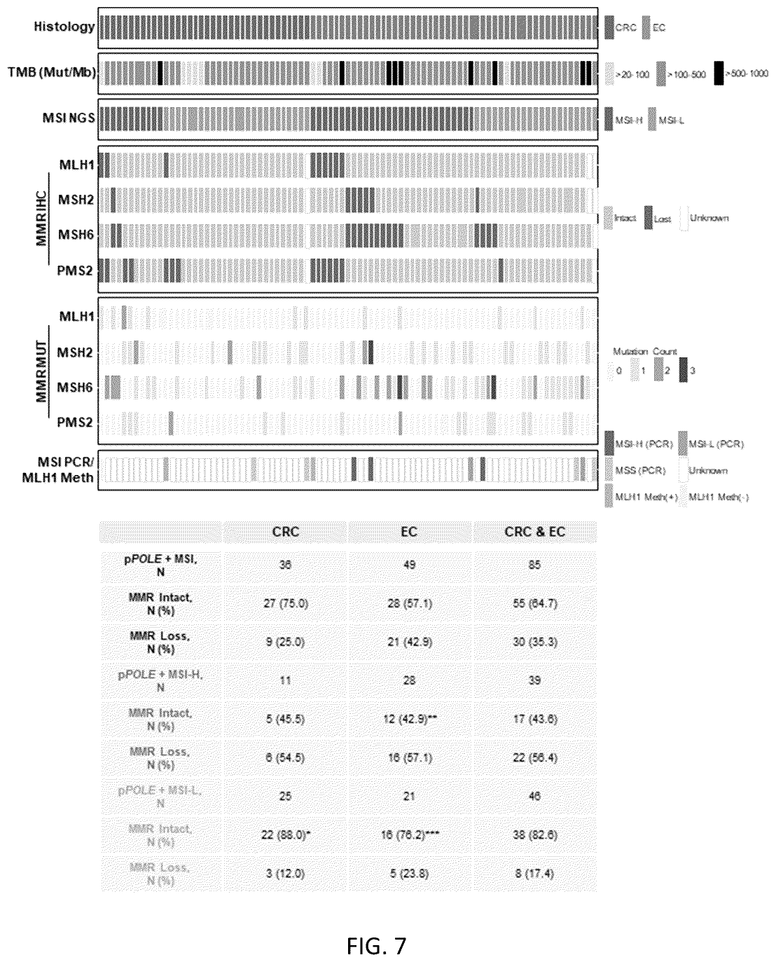

FIG. 7. MMR IHC/MSI PCR Concordance in pPOLE+MSI Samples MMR IHC, MMR mutation status, and additional MSI testing results for pPOLE+MSI samples with available MMR IHC and/or MSI PCR data (CRC n=36, EC n=49). *Includes 1 case called MSS by MSI PCR but lacking MMR IHC data. **Includes 1 case with MMR IHC intact called MSI-Low by MSI PCR. ***Includes 1 case called MSS by MSI PCR but lacking MMR IHC data and 1 case with MMR IHC intact called MSI-equivocal by MSI PCR. CRC, Colorectal Cancer; EC, Endometrial Cancer; MLH1 Meth, MLH1 Promoter Hypermethylation Testing; MMR, Mismatch Repair; MSI, Microsatellite Instability (H-High, Eq-Equivocal); pPOLE, Pathogenic POLE.

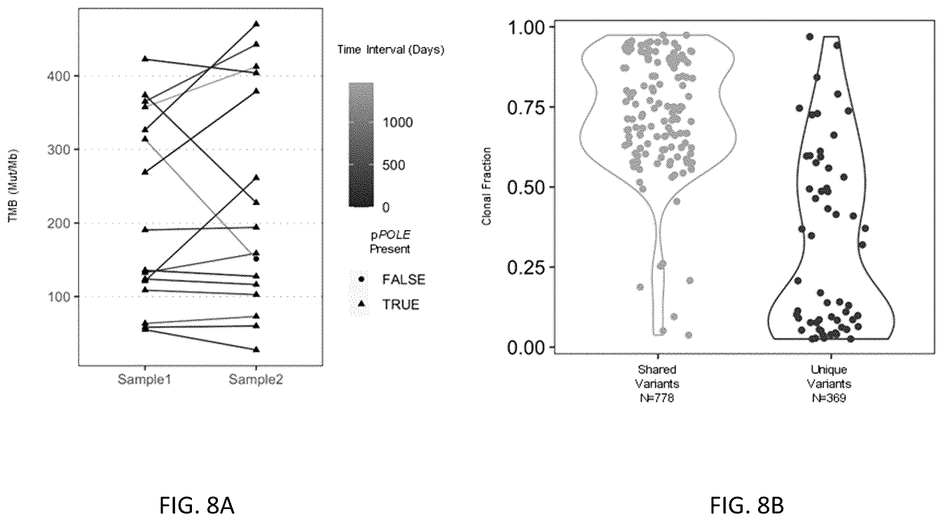

FIGS. 8A-8B. Paired Sample Analysis of Tumors with pPOLE. FIG. 8A: pPOLE status and TMB of paired samples (n=16). FIG. 8B: Clonal fraction (VAF/TP) of non-POLE variants either shared between (n=778) or unique to individual samples (n=369) across sample pairs. pPOLE, Pathogenic POLE; TMB, Tumor Mutational Burden; TP, Computational Tumor Purity; VAF, Variant Allele Fraction.

TABLE 1
Demographic Characteristics of Patients Stratified b-y POLE Mutation Status.
P-values reflect comparisons of subjects with pPOLE versus no POLE mutation.

| | | POLE Mutation Status | | | | | | |
|---|---|---|---|---|---|---|---|---|
| | | pPOLE | | bPOLE | | No POLE Mutation | | p-value |
| Cohort | Overall | 748 | | 18,790 | | 478,231 | | |
| | Tissue Biopsy | 739 | | 17,269 | | 437,957 | | |
| | Liquid Biopsy | 9 | | 1,521 | | 40,274 | | |
| Sex | Female | 441 | (59.0%) | 9,409 | (50.1%) | 257,651 | (53.9%) | 0.006 |
| | Male | 307 | (41.0%) | 9,365 | (49.9%) | 220,350 | (46.1%) | |
| Age | Age 25th Percentile | 46 | | 57 | | 56 | | <0.001 |
| | Age Median (50th Percentile) | 55 | | 66 | | 65 | | |
| | Age 75th Percentile | 64 | | 74 | | 73 | | |
| TMB | TMB-H (≥10 Mut/Mb) | 727 | (97.2%) | 9,204 | (49.0%) | 67,712 | (14.2%) | <0.001 |
| MSI Status | MSS | 481 | (65.6%) | 14,339 | (82.7%) | 414,191 | (94.9%) | <0.001 |
| | MSI-L | 133 | (18.1%) | 609 | (3.5%) | 13,942 | (3.2%) | |
| | MSI-H | 119 | (16.2%) | 2,387 | (13.8%) | 8,343 | (1.9%) | |
| Genomic | AFR | 95 | (13.2%) | 2022 | (11.1%) | 50,499 | (10.9%) | 0.003 |
| Ancestry | AMR | 54 | (7.5%) | 900 | (4.9%) | 25,769 | (5.6%) | |
| | EAS | 38 | (5.3%) | 710 | (3.9%) | 16,917 | (3.7%) | |
| | EUR | 526 | (73.1%) | 14441 | (79.0%) | 364,847 | (78.8%) | |
| | SAS | 7 | (1%) | 205 | (1.1%) | 4,694 | (1.0%) | |
| SBS Signature | POLE | 518 | (72.2%) | 11 | (0.1%) | 19 | (0.0%) | <0.001 |
| | POLE + MMR | 142 | (19.8%) | 47 | (0.5%) | 12 | (0.0%) | |
| | MMR | 40 | (5.6%) | 2,498 | (27.7%) | 13,664 | (15.5%) | |

TMB = Tumor Mutational Burden;
MSI = Microsatellite Instability;
SBS = Single Base Substitution
Genomic ancestry reflects the 1000 Genomes Project super populations:
AFR = African;
AMR = Admixed American;
EAS = East Asian;
EUR = European;
SAS = South Asian

TABLE 2
Summary Of Frequency, Disease Distribution, And Genomic Biomarkers for pPOLE.

| | | | | | | | | | | POLE-Associated |
|---|---|---|---|---|---|---|---|---|---|---|
| Protein | | | Endometrial | | Colon | | Other | | Median TMB | SBS Signature Rate |
| Effect | OncoKB Classification | Total N | Cancer, N (%) | | Cancer, N (%) | | Cancers, N (%) | | (Mut/Mb) | (%) |
| P286R | Likely Oncogenic | 271 | 85 | (31.4%) | 122 | (45.0%) | 64 | (23.6%) | 187.5 | 97.8% |
| V411L | Likely Oncogenic | 188 | 61 | (32.4%) | 71 | (37.8%) | 56 | (29.8%) | 201.3 | 92.0% |
| A456P | Likely Oncogenic | 59 | 16 | (27.1%) | 26 | (44.1%) | 17 | (28.8%) | 155.0 | 94.9% |
| S459F | Likely Oncogenic | 46 | 6 | (13.0%) | 15 | (32.6%) | 25 | (54.3%) | 91.3 | 60.9% |
| S297F | Likely Oncogenic | 40 | 11 | (27.5%) | 13 | (32.5%) | 16 | (40.0%) | 99.5 | 72.5% |
| P436S | Not Listed / Evaluated | 20 | 4 | (20.0%) | 9 | (45.0%) | 7 | (35.0%) | 270.0 | 85.0% |
| A465V | Not Listed / Evaluated | 19 | 2 | (10.5%) | 5 | (26.3%) | 12 | (63.2%) | 231.9 | 78.9% |
| P436R | Likely Oncogenic | 15 | 6 | (40.0%) | 6 | (40.0%) | 3 | (20.0%) | 178.8 | 80.0% |
| M444K | Likely Oncogenic | 13 | 2 | (15.4%) | 8 | (61.5%) | 3 | (23.1%) | 123.8 | 100.0% |
| D275G | Not Listed / Evaluated | 10 | 1 | (10.0%) | 6 | (60.0%) | 3 | (30.0%) | 333.7 | 80.0% |
| N363D | Not Listed / Evaluated | 8 | 5 | (62.5%) | 1 | (12.5%) | 2 | (25.0%) | 255.0 | 87.5% |
| F3675 | Likely Oncogenic | 7 | 3 | (42.9%) | 0 | (0%) | 4 | (57.1%) | 48.8 | 57.1% |
| S461L | Not Listed / Evaluated | 7 | 2 | (28.6%) | 1 | (14.3%) | 4 | (57.1%) | 286.3 | 57.1% |
| S459Y | Likely Oncogenic | 6 | 0 | (0%) | 2 | (33.3%) | 4 | (66.7%) | 165.3 | 83.3% |
| A463D | Not Listed / Evaluated | 5 | 1 | (20.0%) | 2 | (40.0%) | 2 | (40.0%) | 211.3 | 60.0% |
| F367C | Not Listed / Evaluated | 4 | 1 | (25.0%) | 2 | (50.0%) | 1 | (25.0%) | 200.0 | 100.0% |
| H475R | Not Listed / Evaluated | 4 | 0 | (0%) | 1 | (25.0%) | 3 | (75.0%) | 243.2 | 75.0% |
| M295R | Likely Oncogenic | 4 | 1 | (25.0%) | 2 | (50.0%) | 1 | (25.0%) | 183.2 | 75.0% |
| Y458C | Not Listed / Evaluated | 4 | 1 | (25.0%) | 3 | (75.0%) | 0 | (0%) | 300.0 | 75.0% |
| Y458H | Not Listed / Evaluated | 4 | 0 | (0%) | 4 | (100.0%) | 0 | (0%) | 308.8 | 75.0% |
| D275N | Not Listed / Evaluated | 3 | 0 | (0%) | 1 | (33.3%) | 2 | (66.7%) | 357.6 | 66.7% |
| F367V | Likely Oncogenic | 3 | 1 | (33.3%) | 2 | (66.7%) | 0 | (0%) | 429.6 | 100.0% |
| N363K | Not Listed / Evaluated | 2 | 0 | (0%) | 0 | (0%) | 2 | (100.0%) | 85.7 | 50.0% |
| P436H | Not Listed / Evaluated | 2 | 0 | (0%) | 0 | (0%) | 2 | (100.0%) | 197.9 | 50.0% |
| S297Y | Likely Oncogenic | 2 | 1 | (50.0%) | 0 | (0%) | 1 | (50.0%) | 208.8 | 50.0% |
| S461T | Not Listed / Evaluated | 2 | 0 | (0%) | 0 | (0%) | 2 | (100.0%) | 212.8 | 50.0% |
| D275A | Not Listed / Evaluated | 1 | 0 | (0%) | 0 | (0%) | 1 | (100.0%) | 207.1 | 100.0% |
| Y458del | Not Listed / Evaluated | 1 | 1 | (100.0%) | 0 | (0%) | 0 | (0%) | 428.8 | 100.0% |
| Y458F | Likely Oncogenic | 1 | 1 | (100.0%) | 0 | (0%) | 0 | (0%) | 422.6 | 100.0% |

TABLE 3
Dako 22C3 PD-L1 Immunohistochemistry Results For POLE-Mutated
Colorectal and Endometrial Cancers Stratified By pPOLE Status

| | Colorectal | | Endometrial | |
|---|---|---|---|---|
| Dako PD-L1 TPS Category | bPOLE | pPOLE | bPOLE | pPOLE |
| Negative (TPS < 1%) | 400 (75.3%) | 45 (75.0%) | 160 (63.5%) | 40 (70.2%) |
| Positive (TPS ≥ 1%) | 131 (24.7%) | 15 (25.0%) | 92 (36.5%) | 17 (29.8%) |

TPS = Tumor Proportion Score

### Pan-Tumor Landscape of POLE Mutations

A total of 497,769 samples were examined for all classes of POLE variants, including 455,965 tissue biopsies (TBx) and 41,804 liquid biopsies (LBx) (Table 1, FIG. 5). After removal of germline polymorphisms, one or more POLE alterations, including both pathogenic POLE (pPOLE) and benign POLE (bPOLE) alterations were detected in 3.9% (19,538/497,769) of samples. The number of POLE variants per sample ranged from 1-14, with 8.3% (1,627/19,538) of mutated samples harboring multiple variant alleles, resulting in a total of 21,816 mutations. The overall prevalence of POLE mutations was similar for TBx and LBx (3.9% [18,008/455,965] vs 3.7% [1,530/41,804]; P=0.15). A total of 7,404 distinct short variant (i.e., single nucleotide variants [SNVs] and short insertions/deletions [indels]) alleles were observed, 81.1% (6,003/7,404) of which were missense substitutions (FIG. 6A). Individual variant alleles were seen in up to 271 samples, but 53.8% (3,983/7,404) were found in a single subject (FIG. 6B). Mutations involved 97.4% (2,228/2,287) of codons in the canonical POLE peptide (NP_006222.2), with a mean of 3.1 variant alleles per codon, approaching naturally occurring saturation mutagenesis in this real-world cohort (FIGS. 1A-1B).

Sufficient qualifying somatic mutations were present for determination of the most likely single base substitution mutational signatures for 21.5% (98,155/455,965) of tissue samples. For the purposes of this work, two POLE-associated mutational signatures were utilized: one characteristic of POLE mutagenesis, and one characteristic of POLE with co-occurring MMR deficiency (MMRd) (POLE+MMR) (see Methods). A dominant POLE-associated signature (POLE or POLE+MMR) was found in a total of 749 tumors (0.2% of TBx). The POLE-associated mutational signatures had the highest median TMB (158.8 mut/Mb for POLE and 345.1 mut/Mb for POLE+MMR) of all signatures identified in this dataset (FIG. 6C). However, very few POLE variants, even among those within the ExoD, were associated with highly elevated TMB or a POLE-associated signature (FIG. 1C). This pattern highlights the need for accurate classification to identify tumors with POLE-associated ultramutagenesis.

### Application of a POLE-Specific Classification Scheme Identified 29 Pathogenic POLE Variants

A disease model for POLE exonuclease deficiency-mediated ultramutation was applied to identify pPOLE. The 7,404 distinct variant alleles were narrowed to 29 pathogenic variants (Table 2) by excluding 1) germline variants in the gnomAD v2.1 exome database; 2) variants with median TMB<20 mut/Mb across tumors; 3) mutations associated with a POLE-associated SBS signature (see Methods) in less than half of samples in which they were observed; and 4) variants that were only observed to co-occur with another pPOLE variant (FIG. 1D). A total of 748 samples had variants classified as pPOLE (FIG. 1E, Table 2); three samples has two pPOLE. All pPOLE variants were localized to the ExoD (codons 268-471 in NM_006231.4/NP_006222.2), except p.H475R, and were either missense substitutions or in-frame deletions (n=28 and n=1, respectively). It is notable that no pPOLE mutations were predicted to truncate the protein. Among mutations within the ExoD, variants that did not meet criteria for pathogenicity (bPOLE) were 21.4-fold (620/29) more numerous than pPOLE and were seen in 2.1-fold (1,601/757) as many patients, underscoring the importance of variant classification even for mutations within the ExoD.

Unselected POLE mutations were most observed in cancer types associated with high median TMB due to mutagenic exposures (FIG. 1F), including 13.4% (376/2,798) of non-melanoma skin cancers and 7.9% of melanomas (1,107/14,060). In contrast, pPOLE were recurrent in cancers that have been previously associated with POLE-mediated ultramutation, such as those of the endometrium (1.4%, 210/14,776), colon and rectum (0.5%, 302/60,547), and nervous system (0.2%, 34/16,920). The median age of patients with pPOLE was 55 years, which was significantly younger than patients with no POLE mutation (median 65 years, P<0.001) (Table 1). It was notable that more than half (51.4%, 18/35) of pPOLE found in malignancies of the nervous system were in patients under age 30 (FIG. 6D). Compared to the POLE wild type (WT) population, patients with pPOLE were more frequently female (59.0% vs 53.9%, P=0.006), largely explained by the high proportion of EC cases, and more frequently of non-European genomic ancestry (frequencies of AFR, AMR, and EAS subpopulations were elevated compared to POLE WT, P=0.003). Rates of TMB-H (97.2% vs 14.2%), dominant POLE-associated mutational signatures (POLE: 72.2% vs 0.0%, POLE+MMR: 19.8% vs 0.0%), and MSI (MSI-H: 16.2% vs 1.9%, MSI-equivocal: 18.1% vs 3.2%) were all significantly higher in pPOLE cases (P<0.001 for all comparisons) (Table 1). Compared to POLE WT, the presence of bPOLE was associated with biological scenarios with numerous stochastic passenger mutations, including TMB-H, MSI-H, and MMR SBS. Dako 22C3 PD-L1 immunohistochemistry (IHC) results were available for a subset of TBx samples. For CRC and EC, there was no clinically significant difference in the rate of tumor proportion score positive status (≥1% staining) between samples with bPOLE versus pPOLE (Table 3).

pPOLE were observed in both TBx and LBx samples, although the overall frequency was significantly higher in TBx (0.16% [739/455,965] vs 0.02% [9/41,804]; P<0.001). This finding should be interpreted with the context oflow circulating tumor DNA (ctDNA) shed or different clinical populations and ordering patterns for TBx versus LBx, including decreased likelihood of LBx in pPOLE patients due to favorable prognostic profiles. Furthermore, the two matrices had significantly different cohort compositions, with CRC and EC accounting for a higher proportion of TBx than LBx (12.6% [57,345/455,965] vs 7.7% [3,202/41,804] and 3.2% [14,405/455,965] vs 0.9% [371/41,804], respectively; P<0.001 for both comparisons). Nevertheless, pPOLE rates within those cancer types were also lower in LBx.

pPOLE are Associated with Ultramutation

In tissue samples with pPOLE, the median TMB was 186.3 mut/Mb (range 0.0-1,116.4 mut/Mb, FIG. 2A), which was significantly higher than the median for all samples as well samples with any POLE variant (3.5 and 10.0 mut/Mb, respectively; P<0.001 for both comparisons). Median TMB and blood TMB (bTMB) in cancers with pPOLE were comparable (186.3 and 147.9 mut/Mb, respectively; P=0.43). In all cancer types with at least 10 pPOLE, samples with pPOLE had significantly higher TMB than those with bPOLE, as well as POLE WT cases (P<0.001 for all comparisons, FIG. 2B). This was true even in cancer types in which other mutagenic processes are prevalent, including EC (MMRd), CRC (MMRd), and central nervous system (CNS) tumors (MMRd, alkylating agents). Among POLE variants within the ExoD, pPOLE were associated with a significantly higher TMB than bPOLE, including bPOLE observed in the context of a non-POLE mutational process (median 185.7 vs 4.4 and 27.8 mut/Mb, respectively; P<0.001 for both comparisons; FIG. 2C), supporting the positive predictive value of the variant classification scheme.

Pathogenic variant classifications by our functional readout model were compared to AlphaMissense predictions by assessing TMB for variants within the ExoD. AlphaMissense classified 100% (15/15) of pPOLE variants with five or more observations as pathogenic (FIG. 2D; AlphaMissense pathogenicity score range 0.9526-0.9993). However, AlphaMissense also predicted pathogenic status for an additional 40 variants classified as bPOLE by the functional readout model, with a median TMB of 13.8 mut/Mb (IQR 5-55 mut/Mb) for cases harboring these variants.

Though nearly all tumors with pPOLE had high TMB, 2.8% (21/742) had TMB<10 mut/Mb or no TMB point estimate available. Examination of genomic findings in these cases highlighted significantly lower pPOLE variant allele fraction (VAF) compared to pPOLE samples with elevated TMB (median pPOLE VAF of 2.9% vs 25.8%, P<0.001; FIG. 2E). Indeed, 66% (14/21) had tumor purity <20%; while low TMB is not clinically reported in this scenario, it raises the possibility that purity near the limit of detection for TMB can mask patients who may, in fact, have a high neoantigen load and would be expected to respond to ICI. Indeed, in one sample with a pPOLE in which TMB could not initially be reported due to low tumor purity, retesting with macroenrichment to optimize tumor content enabled reporting of a TMB of 150 mut/Mb. This case demonstrates the challenges for accurate TMB estimation in the setting of low tumor purity, but also highlights the potential utility of detection of high specificity pPOLE as a standalone indicator of high TMB. However, four samples with pPOLE and low TMB did have high pPOLE VAF and high tumor purity, raising the possibility that some pPOLE alleles may have variable penetrance.

pPOLE Penetrance Differs Based on Variant Allele and May Depend on Co-Occurring Mismatch Repair Deficiency

The most frequently recurrent pPOLE alleles in this study have been described as pathogenic in the literature including p.P286R (N=271), p.V411L (N=188), p.A456P (N=59), and p.S459F (N=46) (FIG. 1E). However, 16 of 29 (55.2%) predicted pPOLE variants, largely representing more rare alterations, were not classified as pathogenic in the OncoKB21,22 database as of this writing. The majority of pPOLE alleles (72.4% [21/29]) occurred at a codon with at least one other pPOLE allele. However, variants occurring at the same codon did not have uniform pPOLE or bPOLE status. For example, p.P286S is classified as pathogenic in databases and literature and is co-localized with p.P286R, the most common pathogenic allele. While p.P286S was associated with an elevated median TMB of 54.0 mut/Mb (FIG. 3A), 66.6% (10/15) of samples had a UV mutational signature, rather than a POLE-associated signature. Six additional variants (p.P286H, p.P286L, p.D368Y, p.V411M, p.L424I, and p.L424V), many of which were co-localized with a pPOLE, were classified as pathogenic in OncoKB but were classified as bPOLE by the functional readout model.

Variants at codon 424 were examined closely because the functional readout model classified all variants at this locus as bPOLE, although germline p.L424V (c.1270C>G; ClinVar Variation ID: 40046) has been associated with early onset polyposis and CRC [23]. This codon had seven distinct substitutions observed across 84 subjects. Of the recurrent alleles, p.L424F (n=32) and p.L4241 (n=6) werwas excluded due to low median TMB and lack of a consistent association with a POLE SBS signature, respectively. The germline risk allele p.L424V (n=42) was also not associated with elevated median TMB (7.2 mut/Mb); however, closer examination revealed a bimodal distribution (FIG. 3A). While 85.7% (36/42) of samples with this variant had TMB<20 mut/Mb, the other six samples had highly elevated TMB (range 89.6-603.8 mut/Mb) and a POLE-associated SBS signature. Five of the six samples had elevated microsatellite instability (MSI) and POLE+MMR-associated SBS signatures. These findings raise the possibility that some pPOLE might have variable penetrance or that the phenotype might depend on co-occurring MMRd.

Examination of other recurrent variants validated this hypothesis. Two recurrent variants had less than 80% penetrance for TMB≥20 mut/Mb: p.F367S (57.1%, 4/7) and p.S461L (71.4%, 5/7). Seven variants were seen in association with MSI in ≥50% of samples (FIG. 3A): p.D275G (100%, 10/10), p.A463D (100%, 5/5), p.A465V (94.7%, 18/19), p.S461L (85.7%, 6/7), p.N363D (62.5%, 5/8), p.P436S (55.0%, 11/20), and p.S459Y (50.0%, 3/6). This phenomenon led us to further explore the overlap of pPOLE and MSI.

Co-Occurrence of pPOLE and Microsatellite Instability (MSI) Produces a Synergistic Effect on TMB

POLE status interacted with complex biomarkers beyond TMB. Across all cancer types, 34.3% of tissue samples with pPOLE had evidence of co-occurring MSI (16.2% MSI-H and 18.1% MSI-equivocal). Combined pPOLE and MSI was most frequently observed in tumors of the CNS (82.9% of pPOLE samples) but was also seen in more than 25% of pPOLE cases from patients with cancers of the small intestine, prostate, pancreaticobiliary tree, endometrium, and colon/rectum (FIG. 3B). Co-occurrence of pPOLE and MSI produced a synergistic increase in TMB. Whereas samples with pPOLE and no evidence of MSI had a median TMB of 135.7 mut/Mb, pPOLE combined with either MSI-equivocal or MSI-H status resulted in significantly higher median TMBs of 252.5 mut/Mb and 325.6 mut/Mb, respectively (P<0.001 for both comparisons; FIG. 3C). TMB in MSI-H samples was approximately 2-fold higher than would result from an additive effect of pPOLE and MSI. Elevated TMB in tumors with combined pPOLE and MSI was attributable to significant increases in both SBS (median of 130 for MSS versus 250.5 for MSI-equivocal and 275 for MSI-H, P<0.001 for both comparisons) and indels (median of 2 for MSS versus 6 for MSI-equivocal and 21.5 for MSI-H; P<0.001 for both comparisons; FIG. 3D).

MMR protein expression status was extracted from submitted pathology reports in pPOLE+MSI CRC (n=36) and EC (n=49) (FIG. 7). MMR IHC was reported as intact for 75.0% (27/36) of pPOLE+MSI CRC and 57.1% (28/49) of pPOLE+MSI EC. Loss of MMR staining was significantly more common in MSI-H versus MSI-equivocal tumors in both CRC and EC (CRC: 54.5% vs 12.0%, P=0.01; EC: 57.1% vs 23.8%, P=0.02). MMR loss with a corresponding MMR gene mutation (e.g., MSH6 loss+MSH6 mutation) was also more frequent in MSI-H versus MSI-equivocal tumors in both CRC and EC (CRC: 45.5% vs 4.2%, P=0.01; EC: 32.1% vs 20.0%, P-0.51). Interestingly, a sizable proportion of pPOLE+MSI tumors were reported to have intact MMR IHC but harbored pathogenic MMR gene mutations (CRC: 42.9% [MSI-equivocal 45.8% vs MSI-H 36.4%, P=0.72]; EC 50.0% (MSI-equivocal 70.0% vs MSI-H 35.7%, P=0.04]), raising the possibility that some MMR gene mutations may be subclonal in the setting of POLE-associated mutagenesis.

Paired Samples Highlight the Subclonal Nature of Some Variants Arising Due to pPOLE-Associated Mutagenesis

Variant clonality was further explored in sixteen subjects with pPOLE and comprehensive genomic progiling (CGP) on multiple samples collected at distinct time points. Of these 32 samples, 25 (78.1%) had TMB≥100 mut/Mb and all (100%) had TMB≥10 mut/Mb (FIG. 8A). pPOLE status was concordant in all pairs except one from an esophageal cancer patient with 1,240 days between sample collections. While the first timepoint was pPOLE-positive with a TMB of 313.8 mut/Mb, the second sample was pPOLE-negative with 151.3 mut/Mb. Interestingly, a POLE SBS signature was still detected, suggesting loss the pPOLE allele but not the functional readout in the intervening period.

Genomic interpretation of cases with divergent TMBs pointed to clonal heterogeneity of TMB-eligible variants as a likely explanation, supported by the fact that many non-POLE variants were not shared between temporally separated samples from the same patient. Out of 1,147 pathogenic non-POLE alterations in paired samples, 778 variants (67.8%) were found at both timepoints, while 369 (32.2%) variants were unique to a single time point. Variants shared between timepoints had a significantly higher clonal fraction24 than unique variants (median 73.6% vs 18.9%, P<0.001; FIG. 8B). These findings suggest that pPOLE cause ongoing high mutational rates that manifest as many subclonal variants.

pPOLE-Associated Mutagenesis Gives Rise to Incidental Mutations in Clinically Important Genes, Including BRCA

Gene-specific mutation rates were examined to determine whether therapeutically important genes or pathways are affected by pPOLE-associated mutagenesis. EC (FIG. 4A) and CRC (FIG. 4B) TBx with pPOLE had significantly higher gene-specific pathogenic mutation rates, including in therapy-associated genes such as BRCA1 and BRCA2, compared to cases without pPOLE. Samples with BRCA1/2 mutations and no pPOLE mutation had evidence of a homologous recombination-associated genomic scar, as measured by elevated median HRDsig score and rates of HRDsig biomarker-positive status (FIG. 4C), compared to much lower levels for samples with pPOLE, irrespective of BRCA1/2 status (P<0.001 for both metrics). While 52.1% (10,237/19,652) of samples with BRCA1/2 mutations (irrespective of zygosity) were HRDsig+ in the context of pPOLE-negative status, only 1 of 200 (0.5%) BRCA+pPOLE samples was HRDsig+suggesting that POLE mediated ultramutation and HRD are mutually exclusive mechanisms of genomic instability.

### IV. Discussion

Landmark genomic studies established somatic pPOLE mutations as defining drivers of subsets of ultramutated, microsatellite-stable CRC. POLE mutations initially associated with this phenotype were recurrent and localized within or adjacent to the highly conserved exo motifs of the ExoD that are essential for proofreading activity. Subsequently, many somatic POLE mutations, both within and outside the ExoD, have been suggested as pathogenic with variable levels of evidence, and there is no established paradigm for POLE pathogenicity determination. Several studies have ascribed pathogenicity and ICI efficacy to POLE VUS in hypermutated tumors, but treatment responses may be attributable to stochastic factors or independent predictors of immunotherapy response such as elevated TMB due to non-POLE mutagenic processes, including MMRd- or UV-associated mutagenesis. POLE is a large gene (16,609 bp gene length with 49 exons) and is susceptible to accumulation of passenger mutations, as evidenced by the significantly higher TMB for tumors with bPOLE compared to POLE WT. Indeed, near saturation mutagenesis of POLE was observed across 497,769 solid tumors, many of which were associated with non-POLE mutational signatures. This expansive dataset offers an opportunity to use functional readout of the POLE phenotype to confidently identify bPOLE that are not causative for ultramutation and pPOLE that portend favorable prognosis and predict response to ICI.

To address the absence of a unified POLE variant classification scheme, a POLE-specific heuristic model was developed that incorporates multiple genomic features of the POLE-associated ultramutation tumor phenotype and applied it to variants from >490,000 cancers. This approach facilitated identification of 29 POLE variants, including 16 not previously reported as pathogenic, which are considered to be pPOLE and causal of ultramutation. This pathogenicity definition prioritized specificity for association with ultramutation over sensitivity, and additional variants, especially those with potentially lower penetrance of the POLE phenotype were not classified as pPOLE.

It is notable that AlphaMissense, a machine learning classifier for missense variant pathogenicity prediction, classified all recurrent pPOLE as pathogenic, along with 40 additional variants that predominantly had low median TMBs in the cohort. AlphaMissense and other classifiers may yield false positives for POLE mutation effect or may identify modes of pathogenicity that do not produce ultramutation, thus context-specific interpretation of algorithmic predictions is critical because clinical implications of pPOLE are thought to derive from ultramutation.

While variants classified by the model as pPOLE were most frequently observed in EC and CRC, the prevalence of pPOLE was lower in this study compared to The Cancer Genome Atlas (TCGA) EC (1.4% vs 4.6%) and CRC (0.5% vs 2.5%) cohorts. Further, pPOLE prevalence for these cancers was even lower in LBx than in TBx in our dataset. These differences could be explained by a lower propensity for oncologists to seek CGP, particularly LBx-based CGP, for pPOLE cancers since they are likely to have a favorable prognosis. It is also notable that pPOLE were found in many cancer types not previously associated with this phenotype, highlighting an unappreciated therapeutic opportunity. pPOLE were consistently associated with ultramutation across tumor types and TMB was significantly elevated above both tumors with bPOLE and POLE WT status. Importantly, this held true when looking exclusively at ExoD variants, accentuating the importance of phenotype-based classification. Rare tumors harboring pPOLE without accompanying TMB-H status were largely explained by samples with low tumor purity which precluded TMB calculation. Our high specificity model supports a change in practice wherein detection of a pPOLE variant is interpreted as confidently predicting ultramutation, favorable prognosis, and response to ICI even when TMB status is not available as an orthogonal datapoint.

Pathogenic germline POLE alterations predispose to polymerase proofreading-associated polyposis of the colon (PPAP; MIM 615083) and increased risk of colorectal adenocarcinoma, with some reports of increased risk of extracolonic cancers. Interestingly, the most common deleterious germline POLE variant, p.L424V23,41-45, was not classified as pPOLE by our model due to variability of the POLE-associated phenotype in tumors harboring this variant. While most tumors harboring p.L424V had low TMB, 6 of 42 (14.3%) samples exhibited hyper/ultramutation and POLE mutational signatures, with 5 of 6 samples having evidence of MSI and a POLE+MMR mutational signature. These findings suggest that p.L424V, and other germline or somatic variants, may have variable penetrance with dependency on MMRd.

Previous studies speculated that simultaneous POLE exonuclease and MMR deficiency would not be tolerated due to mutation accumulation diminishing the fitness of cancer cells. This hypothesis was initially supported by studies in which pPOLE and MMRd were found to be mutually exclusive, but subsequent reports have highlighted tumors exhibiting concurrent disruption of both mechanisms. It was observed that co-occurrence of pPOLE and MSI is much more common than previously appreciated. Although this could be explained by biases in our CGP cohort which includes a high proportion of advanced and high-stage recurrent cancers compared to other clinical cohorts, a combined phenotype was found in 34.2% of pPOLE tumors overall with a strikingly high incidence (82.9%) in CNS cancers. pPOLE+MSI tumors had even higher TMB than those with pPOLE alone, consistent with a synergistic effect. While certain variants (e.g., p.P286R, p.A456P) rarely co-occurred with MSI, other variants (e.g., p.D275G, p.A465V) were almost exclusively found in the context of MSI-H tumors supporting the postulation that tolerance of combined defects may be allele specific and associated with variable mutator effects of different alterations. Another question arising from observation of pPOLE+MSI tumors concerns the chronology of the repair defects, i.e., whether an initiating POLE proofreading defect resulted in mutation of an MMR gene leading to MMR pathway dysfunction or, conversely, whether MMRd led to acquisition of a pPOLE. Alternatively, capacity for MMR could be overwhelmed by the multitude of variants arising from POL& deficiency, resulting in inability to correct the mounting number of replication errors and cascading to acquisition of additional indel and SNV mutations. Indeed, a sizable proportion of pPOLE+MSI-H or MSI-equivocal EC and CRC did not have evidence of MMR loss by IHC, although concordance between these modalities was very high among POLE WT/bPOLE samples assessed in validation of the MSI biomarker (manuscript submitted). While MSI status could be subclonal and detected via NGS but not IHC, loss of MMR expression may not underlie acquisition of an MSI phenotype in some pPOLE+MSI tumors. A limitation of this analysis is that IHC results were abstracted from pathology reports with IHC performed at different institutions and with heterogeneous methods. It is possible that subclonal loss of MMR staining, an often underrecognized pattern, might have been missed or misinterpreted. Nevertheless, these findings underscore the value of CGP for contextualizing MMR IHC results.

The pPOLE-associated mutator phenotype yields elevated gene-specific mutation rates throughout the exome and the acquisition of many subclonal mutations. Delineating the biological significance of these mutations is critical from a clinical perspective, as the phenotype can yield superfluous alterations in otherwise targetable genes, especially tumor suppressor genes for which many substitutions can create a deleterious effect. Homologous recombination repair (HRR) genes exemplify this scenario because biallelic inactivating mutations confer susceptibility to poly (ADP-ribose) polymerase inhibitors (PARPi). HRDsig is a machine learning classifier that detects copy number features associated with homologous recombination deficiency (HRD) 43. While BRCA1/2 mutations were associated with HRDsig in the context of pPOLE-negative tumors (pPOLE-/BRCA+, ˜50% HRDsig+), this was not the case in the context of pPOLE (pPOLE+/BRCA+, <1% HRDsig+). This supports mutual exclusivity of POLE-mediated ultramutation and HRD. Without a functional readout of HRD in pPOLE tumors, there is no biological basis for pursuing therapy decisions based on incidental BRCA mutations that are likely monoallelic and not predictive of PARPi response. In general, these findings highlight the importance of POLE context for specific interpretation of genomic findings.

Accurate classification of pPOLE has substantial clinical importance and NCCN guidelines for EC, CRC, and small bowel adenocarcinoma underscore assessment of POLE status. The prognostic significance of pPOLE mutations is supported by studies demonstrating reduced recurrence risk in EC, CRC, and glioma, although whether this association is independent of treatment remains unclear. This favorable prognosis may be explained in part by robust anti-tumor immune responses in these tumors as evidenced by increased numbers of tumor-infiltrating CD8+ lymphocytes, as well as expression of cytotoxic T-cell markers and effector cytokines. This immunogenic tumor microenvironment may be a consequence of the multitude of somatic mutations, some proportion of which represent tumor-specific neoantigens. Another hypothesized explanation for improved outcomes with these tumors relates to a potential liability of the mutator phenotype, i.e., the possibility that while acquisition of so many mutations may be beneficial in early tumorigenesis, this may eventually lead to compromised fitness due to the collective effect of an increasing load of mildly deleterious passenger mutations, including mutations in non-cancer associated genes that are essential for cell survival. Confident POLE pathogenicity assignment has become imminently critical with widespread adoption of the Proactive Molecular Risk Classifier for Endometrial Cancer (ProMisE) risk stratification algorithm, which includes a molecular subtype capturing the “POLE ultramutated” group identified in the TCGA study. While some guidance for POLE variant interpretation in this context has already been proffered, we propose that our functional readout-based pPOLE classifications can be reliably integrated into this framework.

The immunogenicity of pPOLE tumors also recommends them for treatment with ICI with initial demonstrations of efficacy in EC, CRC, and other solid tumors (limited by inclusion of tumor types with innate immune sensitivity and existing ICI indications). The majority of pPOLE EC (70%) and CRC (75%) in the study were PD-L1-negative by IHC (Dako 22C3), highlighting the need for CGP (pPOLE status+/−TMB) to identify these patients with a high likelihood of response. In a retrospective multinational study comparing use of ICI in patients with CRC and polymerase proofreading versus MMR deficiency, patients with pPOLE/pPOLD1 had superior outcomes (ORR, PFS, and OS) than patients with MMRd/MSI-H, suggesting pPOLE are predictive of even greater benefit than previously established ICI biomarkers (Ambrosini et al., manuscript accepted). Howitt et al. predicted an ˜15-fold higher and an ˜118-fold higher median number of predicted neoantigens in POLE-mutated EC than in MSI EC and MSS EC tumors, respectively, which may help explain these clinical observations. Additional clinical studies are needed to determine whether co-occurrence of pPOLE and MSI impacts clinical outcomes.

### V. Conclusion

In summary, a phenotypic classification model was developed to support confident pathogenicity assignment of POLE variants causal of ultramutation. Nuanced exploration of >750 samples with pPOLE was performed. This allowed for unique insight into the POLE-associated tumor phenotype, including the subpopulation of tumors with loss of multiple DNA replication fidelity mechanisms (pPOLE+MMRd/MSI).

### EXEMPLARY IMPLEMENTATIONS

Exemplary implementations of the methods and systems described herein include:
1. A method for diagnosing or confirming a diagnosis of disease in a subject, comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and diagnosing or confirming a diagnosis of the disease in the subject based on an indication that the pathogenic POLE (pPOLE) variant is present, with and without an indication of MSI-high status.
2. The method of claim 1, further comprising administering a treatment for the disease to the subject based on the diagnosis or confirmation of a diagnosis of the disease.
3. A method for identifying a subject for treatment of a disease, comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   identifying the subject for treatment of the disease based on an indication that the pathogenic POLE (pPOLE) variant is present, with and without an indication of MSI-high status.
4. A method for predicting a treatment outcome for a subject having a disease, comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   predicting a treatment outcome for the subject based on an indication that the pathogenic POLE (pPOLE) variant is present, with and without an indication of MSI-high status.
5. A method for selecting a treatment for a subject having a disease, the method comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   selecting a treatment for the subject based on an indication that the pathogenic POLE (pPOLE) variant is present, with and without an indication of MSI-high status.
6. A method of treating a subject having a disease, the method comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   responsive to an indication that a pathogenic POLE (pPOLE) variant is present, with and without an indication of MSI-high status, administering a treatment for the disease to the subject;
   thereby treating the subject.
7. A method for adjusting a treatment dose for a subject having a disease, comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   responsive to an indication that a pathogenic POLE (pPOLE) variant is present, with and without an indication of MSI-high status, adjusting the treatment dose for the subject.
8. A method for monitoring disease progression or recurrence in a subject comprising:
   a) determining a first disease status indicator for the subject based on a genomic profile for a first sample obtained from the subject at a first time point, wherein the first disease status indicator indicates:
   (i) a presence of a pathogenic POLE (pPOLE) variant, or
   (ii) a presence of a pathogenic POLE (pPOLE) variant and a microsatellite instability (MSI) status of MSI-high;
   b) determining a second disease status indicator for the subject based on a genomic profile for a second sample obtained from the subject at a second time point, optionally wherein the second time point is after the subject has been treated for a disease, and wherein the second disease status indicator indicates:
   (i) a presence of the pathogenic POLE (pPOLE) variant, or
   (ii) a presence of the pathogenic POLE (pPOLE) variant and a microsatellite instability (MSI) status of MSI-high;
   c) comparing the second disease status indicator to the first disease status indicator; and
   d) determining, based on a change in disease status indicator that indicates the presence of the pPOLE variant in the second sample, that the disease is progressing or reoccurring, thereby monitoring the disease progression or recurrence.
9. The method of claim 8, further comprising determining a variant allele frequence (VAF) associated with the pPOLE variant if the pPOLE variant is determined to be present.
10. The method of claim 8 or claim 9, further comprising selecting a treatment for the disease in response to disease progression or recurrence.
11. The method of claim 10, further comprising administering the treatment to the subject in response to disease progression or recurrence.
12. The method of claim 10 or claim 11, further comprising making a decision to adjust the treatment for the subject in response to disease progression or recurrence.
13. The method of claim 12, wherein the decision is a decision to select a different treatment in response to disease progression or recurrence.
14. The method of claim 12, wherein the decision is a decision to keep the same treatment in response to disease progression or recurrence.
15. The method of any one of claims 10 to 14, further comprising adjusting a dosage of the treatment in response to the disease progression or recurrence.
16. The method of claim 15, further comprising administering the adjusted treatment to the subject.
17. The method of any one of claims 8 to 16, wherein the first time point is before the subject has been treated for a disease, and wherein the second time point is after the subject has been treated for the disease.
18. The method of any one of claims 8 to 17, wherein the subject has a cancer, is at risk of having a cancer, is being routinely tested for cancer, or is suspected of having a cancer.
19. The method of claim 18, wherein the cancer is a solid tumor.
20. The method of claim 18, wherein the cancer is a hematological cancer.
21. The method of any one of claims 1 to 17, wherein the disease is cancer.
22. The method of any one of claims 2 to 17, wherein the treatment comprises an anti-cancer therapy.
23. The method of claim 22, wherein the anti-cancer therapy comprises chemotherapy, radiation therapy, immunotherapy, a targeted therapy, or surgery.
24. The method of any one of claims 2 to 23, wherein the treatment comprises an immune checkpoint inhibitor.
25. The method of claim 24, wherein the immune checkpoint inhibitor comprises an anti-PD-1 or anti-PD-L1 antibody.
26. The method of claim 25, wherein the immune checkpoint inhibitor comprises Nivolumab, Pembrolizumab, Atezolizumab, Cemiplimab, Avelumab, Durvalumab, or any combination thereof.
27. The method of any one of claims 18 to 26, wherein the cancer comprises an endometrial cancer, a colorectal cancer, a non-small cell lung cancer (NSCLC), a squamous NSCLC, a non-squamous NSCLC, a metastatic cutaneous squamous cell carcinoma, a small intestine adenocarcinoma, a glioma, or a metastatic Merkel cell carcinoma.
28. A method for identifying a subject for inclusion in a clinical trial, the method comprising:
   acquiring a genomic profile based on a sample from the subject, wherein the genomic profile is indicative of:
   (i) a presence of a pathogenic POLE (pPOLE) variant, or
   (ii) a presence of a pathogenic POLE (pPOLE) variant and a microsatellite instability (MSI) status of MSI-high; and
   identifying the subject as a candidate for inclusion in the clinical trial based on an indication that the pathogenic POLE (pPOLE) variant is present, with and without an indication of MSI-high status.
29. The method of any one of claims 1 to 28, wherein an indication of the presence of a pathogenic POLE (pPOLE) variant in the genomic profile is based on an analysis of sequence read data derived from the sample from the subject.
30. The method of claim 29, wherein the presence of a pathogenic POLE (pPOLE) variant is detected in the sequence read data using one or more processors and a variant calling algorithm.
31. The method of any one of claims 1 to 30, wherein the pPOLE variant comprises a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.
32. The method of any one of claims 1 to 31, wherein an indication of MSI-high status in the genomic profile is based on an analysis of sequence read data for a plurality of microsatellite loci in the sample.
33. The method of any one of claims 1 to 32, wherein an indication of MSI-high status is indicative of a deficient DNA mismatch repair mechanism in the sample.
34. The method of any one of claims 1 to 33, further comprising applying an indication that a pPOLE variant is present in the sample as a diagnostic value associated with the sample.
35. The method of any one of claims 1 to 34, wherein the genomic profile comprises results from a comprehensive genomic profiling (CGP) test, a gene expression profiling test, a cancer hotspot panel test, a DNA methylation test, a DNA fragmentation test, an RNA fragmentation test, or any combination thereof.
36. The method of claim 35, wherein the genomic profile for the subject further comprises results from a nucleic acid sequencing-based test.
37. The method of any one of claims 1 to 36, wherein the sample is a tissue sample derived from the subject.
38. The method of any one of claims 1 to 36, wherein the sample is a liquid biopsy or hematological biopsy sample derived from the subject.
39. The method of claim 38, wherein the sample is a liquid biopsy sample comprising blood, plasma, cerebrospinal fluid, sputum, stool, urine, or saliva.
40. The method of claim 38, wherein the sample is a liquid biopsy sample and comprises circulating tumor cells (CTCs).
41. The method of claim 38, wherein the sample is a liquid biopsy sample and comprises cell-free DNA (cfDNA).
42. The method of claim 41, wherein all or a portion of the cell-free DNA (cfDNA) comprises circulating tumor DNA (ctDNA).
43. A method comprising:
   providing a plurality of nucleic acid molecules obtained from a sample from a subject suspected of having or determined to have cancer;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, using one or more processors, sequence read data for the plurality of sequence reads;
   analyzing the sequence read data, using the one or more processors, to determine:
      (i) a presence of a pathogenic POLE (pPOLE) variant, or
      (ii) a presence of a pathogenic POLE (pPOLE) variant and a microsatellite instability (MSI) status of MSI-high; and
   responsive to an indication that a pathogenic POLE (pPOLE) variant is present, with and without an indication of MSI-high status, administering an anti-cancer therapy to the subject.
44. The method of claim 43, wherein the subject has a cancer, is at risk of having a cancer, is being routinely tested for cancer, or is suspected of having a cancer.
45. The method of claim 43 or claim 44, wherein the pPOLE variant comprises a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.
46. The method of any one of claims 43 to 45, wherein the anti-cancer therapy comprises an immune checkpoint inhibitor.
47. The method of claim 46, wherein the immune checkpoint inhibitor comprises an anti-PD-1 or anti-PD-L1 antibody.
48. The method of claim 47, wherein the immune checkpoint inhibitor comprises Nivolumab, Pembrolizumab, Atezolizumab, Cemiplimab, Avelumab, Durvalumab, or any combination thereof.
49. The method of any one of claims 44 to 48, wherein the cancer comprises an endometrial cancer, a colorectal cancer, a non-small cell lung cancer (NSCLC), a squamous NSCLC, a non-squamous NSCLC, a metastatic cutaneous squamous cell carcinoma, or a metastatic Merkel cell carcinoma.
50. The method of any one of claims 44 to 48, wherein the cancer comprises a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
51. The method of any one of claims 1 to 50, further comprising obtaining the sample from the subject.
52. The method of any one of claims 1 to 51, wherein the sample comprises a tissue biopsy sample, a liquid biopsy sample, or a normal control.
53. The method of claim 52, wherein the sample is a liquid biopsy sample and comprises blood, plasma, cerebrospinal fluid, sputum, stool, urine, or saliva.
54. The method of claim 52, wherein the sample is a liquid biopsy sample and comprises circulating tumor cells (CTCs).
55. The method of claim 54, wherein the sample is a liquid biopsy sample and comprises cell-free DNA (cfDNA).
56. The method of claim 55, wherein the cell-free DNA (cfDNA) or a portion thereof comprises circulating tumor DNA (ctDNA).
57. The method of any one of claims 43 to 56, wherein the plurality of nucleic acid molecules comprises a mixture of tumor nucleic acid molecules and non-tumor nucleic acid molecules.
58. The method of claim 57, wherein the tumor nucleic acid molecules are derived from a tumor portion of a heterogeneous tissue biopsy sample, and the non-tumor nucleic acid molecules are derived from a normal portion of the heterogeneous tissue biopsy sample.
59. The method of claim 57, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
60. The method of any one of claims 43 to 59, wherein the one or more adapters comprise amplification primers, flow cell adaptor sequences, substrate adapter sequences, or sample index sequences.
61. The method of any one of claims 43 to 60, wherein the captured nucleic acid molecules are captured from the amplified nucleic acid molecules by hybridization to one or more bait molecules.
62. The method of claim 61, wherein the one or more bait molecules comprise one or more nucleic acid molecules, each comprising a region that is complementary to a region of a captured nucleic acid molecule.
63. The method of any one of claims 43 to 62, wherein amplifying nucleic acid molecules comprises performing a polymerase chain reaction (PCR) amplification technique, a non-PCR amplification technique, or an isothermal amplification technique.
64. The method of any one of claims 43 to 63, wherein the sequencing comprises use of a massively parallel sequencing (MPS) technique, whole genome sequencing (WGS), whole exome sequencing, targeted sequencing, direct sequencing, or Sanger sequencing technique.
65. The method of claim 64, wherein the sequencing comprises massively parallel sequencing, and the massively parallel sequencing technique comprises next generation sequencing (NGS).
66. The method of any one of claims 43 to 65, wherein the sequencer comprises a next generation sequencer.
67. The method of any one of claims 43 to 66, wherein one or more of the plurality of sequencing reads overlap one or more gene loci within one or more subgenomic intervals in the sample.
68. The method of claim 67, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
69. The method of claim 67 or claim 68, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
70. The method of any one of claims 43 to 69, further comprising generating, by the one or more processors, a report indicating the presence or absence of a pathogenic POLE (pPOLE) variant and/or a microsatellite instability (MSI) status for the sample.
71. The method of claim 70, further comprising transmitting the report to a healthcare provider.
72. The method of claim 71, wherein the report is transmitted via a computer network or a peer-to-peer connection.
73. The method of any one of claims 1 to 72, wherein determination of microsatellite instability (MSI) status comprises:
   identifying, using one or more processors, a set of microsatellite loci from a plurality of microsatellite loci based on a coverage requirement;
   applying, by the one or more processors, a set of sequence-based exclusion criteria to the set of microsatellite loci to identify a subset of the set of microsatellite loci;
   determining, by the one or more processors, a microsatellite instability (MSI) score for the sample based on a number of microsatellite loci in the set and a number of microsatellite loci in the subset;
   comparing, by the one or more processors, the MSI score to a predetermined threshold; and
   determining an MSI status of high microsatellite instability (MSI-high) for the sample if the MSI score is greater than or equal to the threshold.
74. The method of claim 73, wherein an indication of MSI-high status is indicative of a deficient DNA mismatch repair mechanism in the sample.
75. The method of claim 73 or claim 74, wherein the sample is a liquid biopsy sample or a hematological sample, and wherein the plurality of microsatellite loci comprises at least 1,000 loci.
76. The method of claim 73 or claim 74, wherein the sample is a tissue sample, and wherein the plurality of microsatellite loci comprises at least 2,000 loci.
77. The method of any one of claims 73 to 76, wherein the microsatellite loci comprise alleles having mononucleotide, dinucleotide, or trinucleotide repeat sequences.
78. The method of any one of claims 73 to 77, wherein the sample is a tissue sample, and each microsatellite locus in the plurality of microsatellite loci comprises an allele having an overall length of at least 6 base pairs and less than 30 base pairs.
79. The method of any one of claims 73 to 78, wherein each microsatellite locus in the plurality of microsatellite loci comprises an allele having a mononucleotide, dinucleotide, or trinucleotide repeat sequence at a minimum of 5× repeats, and having a total length of less than 50 base pairs.
80. The method of any one of claims 73 to 79, wherein the coverage requirement is at least 75×, 100×, 150×, 150×, 200×, or 250×.
81. The method of any one of claims 73 to 80, wherein the coverage requirement is locus-dependent.
82. The method of any one of claims 73 to 81, wherein applying the set of sequence-based exclusion criteria comprises excluding, from the set of microsatellite loci, a microsatellite locus that comprises an allele having an allele frequency below an allele frequency requirement.
83. The method of claim 82, wherein the allele frequency requirement is at least 1%, at least 2%, at least 3%, at least 4%, at least 5%, at least 6%, at least 7%, at least 8%, at least 9%, or at least 10%.
84. The method of any one of claims 73 to 81, wherein applying the set of sequence-based exclusion criteria comprises excluding, from the set of microsatellite loci, a microsatellite locus that comprises an erroneous allele sequence according to a statistical model.
85. The method of any one of claims 73 to 81, wherein applying the set of sequence-based exclusion criteria comprises:
   comparing a particular allele at a particular microsatellite locus from the set of microsatellite loci to a reference database of sequencing errors; and
   excluding the particular microsatellite locus from the set of microsatellite loci if the particular allele corresponds to a known sequencing error.
86. The method of claim 85, wherein the particular microsatellite locus is excluded if the particular allele is an allele of less than 10 base pairs in length and the particular allele has an allele frequency less than or equal to a mean allele frequency plus two standard deviations for the particular allele in the reference database of sequencing errors.
87. The method of claim 85 or claim 86, wherein the particular microsatellite locus is excluded if the particular allele is an allele of greater than or equal to 10 base pairs in length and the particular allele has an allele frequency less than or equal to a mean allele frequency plus three standard deviations for the particular allele in the reference database of sequencing errors.
88. The method of any one of claims 73 to 81, wherein applying the set of sequence-based exclusion criteria comprises comparing a particular allele at a particular microsatellite locus to one or more databases; and excluding the particular of microsatellite locus if the particular allele corresponds to a known germline allele.
89. The method of any one of claims 73 to 81, wherein applying the set of sequence-based exclusion criteria comprises comparing a particular allele at a particular microsatellite locus to one or more databases; and excluding the particular microsatellite locus if the particular allele is equal in repeat length to a repeat length for the particular allele in the one or more databases, equal in overall length to an overall length for the particular allele in a reference human genome database, or equal in number of repeats to a number of repeats for the particular allele in the one or more databases.
90. The method of any one of claims 73 to 89, wherein the set of sequence-based exclusion criteria is locus-dependent.
91. The method of any one of claims 73 to 90, wherein the MSI score is calculated by comparing the number of microsatellite loci in the subset to the number of microsatellite loci in the set.
92. A system comprising:
   one or more processors; and
   a memory communicatively coupled to the one or more processors and configured to store instructions that, when executed by the one or more processors, cause the system to perform the method of any one of claims 1 to 91.
93. A non-transitory computer-readable storage medium storing one or more programs, the one or more programs comprising instructions, which when executed by one or more processors of a system, cause the system to perform the method of any one of claims 1 to 91.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables.

Various modifications in form and detail of the embodiments of the invention will be apparent to a person skilled in the art. It is therefore contemplated that the invention shall also cover any such modifications, variations and equivalents.

What is claimed is:

1. A method for treating a subject for cancer, the method comprising:
providing a plurality of nucleic acid molecules obtained from a sample from a subject suspected of having, at risk of having, or determined to have cancer;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, using one or more processors, sequence read data for the plurality of sequence reads;
analyzing the sequence read data, using the one or more processors, to determine:
(i) a presence of a pathogenic POLE (pPOLE) variant, or
(ii) a presence of a pathogenic POLE (pPOLE) variant and a microsatellite instability (MSI) status of MSI-high; and
responsive to an indication that a pathogenic POLE (pPOLE) variant is present, with and without an indication of MSI-high status, identifying the subject as a candidate for anti-cancer therapy.

2. The method of claim 1, further comprising administering an anti-cancer therapy to the subject.

3. The method of claim 1, wherein the pPOLE variant comprises a P286R, V411L, A456P, S459F, S297F, P436S, A465V, P436R, M444K, D275G, N363D, F367S, S461L, S459Y, A463D, F367C, H475R, M295R, Y458C, Y458H, D275N, F367V, N363K, P436H, S297Y, S461T, D275A, Y458del, or Y458F variant in the POLE gene.

4. The method of claim 1, wherein the anti-cancer therapy comprises an immune checkpoint inhibitor.

5. The method of claim 4, wherein the immune checkpoint inhibitor comprises an anti-PD-1 or anti-PD-L1 antibody.

6. The method of claim 5, wherein the immune checkpoint inhibitor comprises Nivolumab, Pembrolizumab, Atezolizumab, Cemiplimab, Avelumab, Durvalumab, or any combination thereof.

7. The method of claim 1, wherein the cancer comprises an endometrial cancer, a colorectal cancer, a non-small cell lung cancer (NSCLC), a squamous NSCLC, a non-squamous NSCLC, a metastatic cutaneous squamous cell carcinoma, or a metastatic Merkel cell carcinoma.

8. The method of claim 1, wherein the sample is a liquid biopsy sample and comprises blood, plasma, cerebrospinal fluid, sputum, stool, urine, saliva, circulating tumor cells (CTCs), cell-free DNA (cfDNA), or circulating tumor DNA (ctDNA).

9. The method of claim 1, wherein determination of microsatellite instability (MSI) status comprises:
identifying, using one or more processors, a set of microsatellite loci from a plurality of microsatellite loci based on a coverage requirement;
applying, by the one or more processors, a set of sequence-based exclusion criteria to the set of microsatellite loci to identify a subset of the set of microsatellite loci;
determining, by the one or more processors, a microsatellite instability (MSI) score for the sample based on a number of microsatellite loci in the set and a number of microsatellite loci in the subset;
comparing, by the one or more processors, the MSI score to a predetermined threshold; and
determining an MSI status of high microsatellite instability (MSI-high) for the sample if the MSI score is greater than or equal to the threshold.

10. The method of claim 9, wherein an indication of MSI-high status is indicative of a deficient DNA mismatch repair mechanism in the sample.

11. The method of claim 9, wherein each microsatellite locus in the plurality of microsatellite loci comprises an allele having a mononucleotide, dinucleotide, or trinucleotide repeat sequence at a minimum of 5× repeats, and having a total length of less than 50 base pairs.

12. The method of claim 9, wherein the coverage requirement is at least 75×, 100×, 150×, 150×, 200×, or 250× and is locus-dependent.

13. The method of claim 9, wherein applying the set of sequence-based exclusion criteria comprises excluding, from the set of microsatellite loci, a microsatellite locus that comprises an allele having an allele frequency below an allele frequency requirement.

14. The method of claim 9, wherein applying the set of sequence-based exclusion criteria comprises excluding, from the set of microsatellite loci, a microsatellite locus that comprises an erroneous allele sequence according to a statistical model.

15. The method of claim 9, wherein applying the set of sequence-based exclusion criteria comprises:
comparing a particular allele at a particular microsatellite locus from the set of microsatellite loci to a reference database of sequencing errors; and
excluding the particular microsatellite locus from the set of microsatellite loci if the particular allele corresponds to a known sequencing error.

16. The method of claim 9, wherein applying the set of sequence-based exclusion criteria comprises comparing a particular allele at a particular microsatellite locus to one or more databases; and excluding the particular of microsatellite locus if the particular allele corresponds to a known germline allele.

17. The method of claim 9, wherein applying the set of sequence-based exclusion criteria comprises comparing a particular allele at a particular microsatellite locus to one or more databases; and
excluding the particular microsatellite locus if the particular allele is equal in repeat length to a repeat length for the particular allele in the one or more databases, equal in overall length to an overall length for the particular allele in a reference human genome database, or equal in number of repeats to a number of repeats for the particular allele in the one or more databases.

18. The method of claim 9, wherein the set of sequence-based exclusion criteria is locus-dependent.

19. A system comprising:
one or more processors; and
a memory communicatively coupled to the one or more processors and configured to store instructions that, when executed by the one or more processors, cause the system to:
receive sequence read data for a plurality of sequence reads derived from a sample from a subject suspected of having, at risk of having, or determined to have cancer;
analyze the sequence read data to determine:
(i) a presence of a pathogenic POLE (pPOLE) variant, or
(ii) a presence of a pathogenic POLE (pPOLE) variant and a microsatellite instability (MSI) status of MSI-high; and
output, based on a determination that a pathogenic POLE (pPOLE) variant is present, with and without an indication of MSI-high status, an identification of the subject as a candidate for anti-cancer therapy.

20. A non-transitory computer-readable storage medium storing one or more programs, the one or more programs comprising instructions, which when executed by one or more processors of a system, cause the system to:
receive sequence read data for a plurality of sequence reads derived from a sample from a subject suspected of having, at risk of having, or determined to have cancer;
analyze the sequence read data to determine:
(i) a presence of a pathogenic POLE (pPOLE) variant, or
(ii) a presence of a pathogenic POLE (pPOLE) variant and a microsatellite instability (MSI) status of MSI-high; and
output, based on a determination that a pathogenic POLE (pPOLE) variant is present, with and without an indication of MSI-high status, an identification of the subject as a candidate for anti-cancer therapy.
